(19) United States
(12) Patent Application Publication
(10) Pub. No.: US 2021/0298566 A1
(43) Pub. Date: Sep. 30, 2021

(54) DISTAL TIP OF A MULTI CAMERA MEDICAL IMAGING DEVICE

(71) Applicant: 270 SURGICAL LTD, Netanya (IL)

(72) Inventors: Avraham LEVY, Kfar Shmaryahu (IL); Golan SALMAN, Atlit (IL); Amram AIZENFELD, Ramot Menashe (IL); Leonid KRIVOPISK, Nesher (IL)

(21) Appl. No.: 17/263,920

(22) PCT Filed: Jul. 28, 2019
(86) PCT No.: PCT/IL2019/050851

§ 371 Date: Jan. 28, 2021

Related U.S. Application Data
(60) Provisional application No. 62/790,493, filed on Jan. 10, 2019.
(60) Provisional application No. 62/713,005, filed on Aug. 1, 2018.

(51) Int. Cl. A61B 1/00; A61B 1/06; A61B 1/05

(52) U.S. Cl. CPC A61B 1/0019; A61B 1/0676; A61B 1/00105; A61B 1/00183; A61B 1/0684; A61B 1/05; A61B 1/00181

(57) ABSTRACT

The subject matter discloses a multi camera medical imaging device comprising a distal tip connected to a rigid shaft, comprising a front modular imaging unit having a front optical gear that captures a front field of view, a primary modular imaging unit adapted to house the front modular imaging unit, said primary modular imaging unit comprises a longitudinal opening located on a lateral side of the primary modular imaging unit, in parallel to a longitudinal axis of the primary modular imaging unit and a secondary modular imaging unit comprising a second side optical gear, wherein the second side optical gear captures a field of view, wherein the second side optical gear is located in parallel to a longitudinal axis of the secondary modular imaging unit, and wherein said secondary modular imaging unit is adapted to close the longitudinal opening and function as a bracket which seals the longitudinal opening.

### FIELD OF THE INVENTION

The present invention relates generally to the field of medical instruments designed to capture images from inside a lumen.

### BACKGROUND OF THE INVENTION

Endoscopes are medical devices which have been utilized to perform operations in the internal organs of the body through small incision in skin. Over the years such medical devices have utilized to perform operations and capture images in internal organs of a body. Such medical devices have been developed and categorized according to specific applications, such as laparoscopes, arthroscopes, cystoscopes, ureteroscopes, hysterectomy and others. In some cases, these medical devices can enter the body through small incisions in the body. Typically, such operations require the aid of a camera and in some cases, more than one camera. As of today, there are multiple different types of flexible endoscopes, rigid and semi-rigid endoscopes, depending on the area/lumen in which the device is used and the procedure's type. As a result of that, to enable the required field of view during the medical procedure the scope has to be directed to the region of interest constantly.

A standard rigid and semi-rigid endoscope is likely to be designed as an elongated tubular member in which the optical gear is located at its distal tip, as well as all the electrical circuitry. In most cases, the flexible endoscopes are inserted into the patient's body via natural orifices, while rigid and semi-rigid endoscopes typically inserted into the body via small incisions, which can be about 11 millimetres or less, made in the patient's body.

### SUMMARY OF THE INVENTION

The subject matter in the present invention discloses a medical imaging rigid scope comprising a distal tip formed of a front modular imaging unit, a primary modular imaging unit and a secondary modular imaging unit. The front modular imaging unit may comprise a front optical gear designed to capture fields of view required for the operations of the medical imaging rigid scope. Such operations may comprise the utilization of a medical imaging device in the activities required at any medical procedure aided by more than one camera. The medical imaging device may further be configured to afford a user to manipulate by a handle the distal tip of the device, and/or to be manipulated by a robotic arm or robotic gripping means.

The primary modular imaging unit may be adapted to house the front modular imaging unit at the distal end of the primary modular imaging unit. In some cases, the primary modular imaging unit may also be adapted to connect to a rigid shaft. The rigid shaft may contain electrical means for the communications between the electrical equipment and an optical gear utilized by the medical imaging rigid scope. For example, such communications may be, digital video signals, electrical signals required for the operation of the optical gear, and the like. In some cases, electrical power may also be conducted by wiring located in the rigid shaft.

In some cases, the front modular imaging unit may seal the front end of the primary modular imaging unit and prevent leaks or ingresses from the outer environment into the medical imaging rigid scope. The primary modular imaging unit may comprise a longitudinal opening located on a lateral side of the primary modular imaging. In some cases, the longitudinal opening may be utilized to assemble and connect the optical gear and the equipment of the primary modular imaging. In some cases, the optical gear of the primary modular imaging unit may be connected to a circuit board located within the primary modular imaging, on a foldable arm. The foldable arm may be designed to be put into said primary modular imaging unit, in a folded position. The primary modular imaging unit further may comprise a foldable circuit board connected to the optical gear of said primary modular imaging unit. The primary modular imaging unit further may comprise illuminator circuit board comprising illumination bodies.

The secondary modular imaging unit may also comprise a second side optical gear configured to capture a field of view required for the operation of the medical imaging rigid scope. The secondary modular imaging unit is adapted to be mounted on the primary modular imaging unit and thereby close the longitudinal opening. Thus, the secondary modular imaging unit may be in parallel to a longitudinal axis of the secondary modular imaging unit and function as a bracket which seals the longitudinal opening. The secondary modular imaging unit may also comprise dedicated margins adapted to be attached to the longitudinal opening of the primary modular imaging, unit.

The secondary modular imaging unit may comprise protruding elements located on the margins of the secondary modular imaging. The protruding elements may be utilized to connect and fasten the secondary modular imaging unit to the primary modular imaging unit. The secondary modular imaging unit may further comprise a foldable circuit board connected to the optical gear of said secondary modular imaging unit. The secondary modular imaging unit may also comprise illuminator circuit board comprising illumination bodies.

In some cases, the front modular imaging unit of the medical imaging rigid scope may comprise illuminator circuit board comprising illumination bodies.

In some embodiments of the disclosed subject matter, the primary modular imaging unit may further comprise a first side optical gear configured to capture a field of view required for the operation of the multi camera medical imaging device. In such cases, the first side optical gear may be mounted on a first side foldable circuit board comprising a foldable arm and designed to be put into said primary modular imaging unit, in a folded position. The first side foldable circuit board can also be positioned in parallel to the primary modular imaging unit longitudinal axis. The foldable circuit board on which the first side optical gear is mounted may extend outwards from said primary modular imaging unit into said rigid shaft. The primary modular imaging unit further comprises a first side illuminator electronic circuit board designed to host first side illumination modules.

The subject matter disclosed in the present invention is a multi-camera medical imaging device designed for obtaining a panoramic view composed by more than one camera. The multi camera medical imaging device embodied in the disclosed subject matter comprises a distal tip adapted to be connected to a rigid shaft of the multi camera imaging device and a front modular imaging unit comprising a front optical gear. The distal tip of the multi camera medical imaging device comprises a front optical gear equipped with a front lens assembly configured to capture a front field of view required for the operations of the multi camera medical imaging device. The disclosed subject matter may also comprise a primary modular imaging unit comprising a first side optical gear. The first side optical gear can be configured to comprise a first side lens assembly characterized with a first direction of view, wherein the first side lens assembly is adapted to capture a first side field of view. In some cases, the distal tip of the multi camera medical imaging device is designed such that, the first direction of view of the first lens assembly can be tilted with a first side angle adapted to provide an overlap view between the front field of view and the first side lens assembly at an overlap distance.

In some embodiments of the disclosed subject matter the first side optical gear is located within the primary modular imaging, unit, and wherein the primary modular imaging unit comprises a first side niche located on an external surface of the primary modular imaging unit and in parallel to a longitudinal axis of the first modular imaging unit.

In some embodiments of the disclosed subject matter the distal tip comprises a secondary modular imaging unit comprising a second side optical gear, wherein the second side optical gear comprises a second side lens assembly characterized with a second direction of view. The second side lens assembly can be configured to capture a second side field of view. In some cases, the distal tip of the multi camera medical imaging device is designed such that the second direction of view is tilted with a second side angle adapted to provide an overlap view between said front field of view and the second side lens assembly at overlap distance.

In one aspect of the disclosed subject matter, the multi camera medical imaging device the first side angle is between 85 to 90 degrees relative to a longitudinal axis of the multi camera medical imaging device. In one aspect of the disclosed subject matter the second side angle is between 85 to 90 degrees relative to a longitudinal axis of the multi camera medical imaging device.

In one aspect of the disclosed subject matter the first overlap distance and said second overlap distance are between 23 to 90 millimeters. In one aspect of the disclosed subject matter the first overlap distance and said second overlap distance are between 78 to 90 millimeters. In one aspect of the disclosed subject matter the first overlap distance and said second overlap distance are between 60 to 64 millimeters. In one aspect of the disclosed subject matter the first overlap distance and said second overlap distance are between 48 to 60 millimeters. In one aspect of the disclosed subject matter the first overlap distance and said second overlap distance are between 42 to 48 millimeters. In one aspect of the disclosed subject matter the first overlap distance and said second overlap distance are between 23 to 33 millimeters. In one aspect of the disclosed subject matter the first overlap distance equals to said second overlap distance.

In some embodiments of the disclosed subject matter the second side optical gear may be located within a secondary modular imaging unit, and the secondary modular imaging unit may comprise a second side niche located on an external surface of the secondary modular imaging unit and in parallel to a longitudinal axis of the secondary modular imaging unit. In some cases, the second side niche comprises a second side tilted camera platform configured to house a second side camera optical window, wherein the second side camera optical window is abutted to the second side camera. In such cases, the tilted camera platform inclines outward from said second side niche. In one aspect of the disclosed subject matter the second side niche has a depth of essentially between 0.1 and 0.8 millimeters.

In some embodiments of the disclosed subject matter the secondary modular imaging unit may also comprise the second side optical gear. Such a second side optical gear can comprise a second side camera comprising a second side lens assembly and a second side sensor. The second side lens assembly can be configured to capture light from the second side lens assembly and convert the captured light to electrical signals in form of current. In one aspect of the disclosed subject matter the second side angle is essentially between 85 to 90 degrees relative to a longitudinal axis of the multi camera medical imaging device. The secondary modular imaging unit also comprises a second side foldable circuit board having a second side planar rigid circuit board section and a main section. The second side planar rigid section is designed to hold the second side camera required for imaging operation of said multi camera medical imaging device.

The secondary modular imaging unit also comprises a second side illuminator electronic circuit board which can hold a set of one or more illumination modules. The second side printed circuit board is positioned in parallel to the second camera defined by the second angle. The secondary modular imaging unit also comprises a protruding element positioned at a distal end of said secondary modular imaging unit for securing the secondary modular imaging unit within a primary modular imaging unit of said device. The primary modular imaging unit further comprises the first side optical gear, wherein such a first side optical gear may comprise a first side camera comprises a first side lens assembly and a first side sensor. The first side lens assembly can be configured to capture light from the first side lens assembly and convert the captured light to electrical signals in form of current. The first side camera is characterized with a direction of view essentially perpendicular to a longitudinal axis of the multi camera medical imaging device.

In some embodiments of the disclosed subject matter the multi camera medical imaging device comprises a first side lens assembly tilted to a first angle adapted to provide an overlap view between said first side lens assembly and a front lens assembly at a first overlap distance. In such embodiments, the multi camera medical imaging device also comprises a second side lens assembly tilted to a second angle adapted to provide an overlap view between said second side lens assembly and the front lens assembly at a second overlap distance.

In one aspect of the disclosed subject matter the first angle is between 85 and 90 degrees relative to a longitudinal axis of the multi camera medical imaging device. In one aspect of the disclosed subject matter the second angle is between 85 and 90 degrees relative to a longitudinal axis of the multi camera medical imaging device.

In one aspect of the disclosed subject matter the first overlap distance is between 60 and 64 millimeters. In one aspect of the disclosed subject matter the second overlap distance is between 60 to 64 millimeters. In one aspect of the disclosed subject matter the second overlap distance is between 48 to 60 millimeters

In one aspect of the disclosed subject matter the first overlap distance is between 42 to 48 millimeters. In one aspect of the disclosed subject matter the second overlap distance is between 42 to 48 millimeters. In one aspect of the disclosed subject matter the first overlap distance is between 42 to 64 millimeters. In one aspect of the disclosed subject matter the second overlap distance is between 42 to 64 millimeters. In one aspect of the disclosed subject matter the first overlap distance equals to said second overlap distance.

In some embodiments of the disclosed subject matter the distal tip of the multi camera medical imaging device may comprise a side lens assembly tilted to an angle adapted to provide an overlap view between the side lens assembly and a front lens assembly at as working distance. The distal tip may also comprise: a secondary modular imaging unit comprising a second optical gear adapted to provide a second side view for the secondary modular imaging unit device. The secondary modular imaging unit may comprise the second side niche along a longitudinal axis of said secondary modular imaging unit device. In some cases, the second niche may comprise a tilted second side camera platform adapted to hold a second side camera optical window which covers and protects a second side lens assembly. The second side niche may also comprise a second side illuminator window and a second side illuminator window located along the longitudinal axis of the secondary modular imaging unit, on both sides of said second side camera optical window. In such cases, the second side niche may be characterized with a niche depth in a range of 0.1 to 0.8 millimeters relatively to a lateral side of said secondary modular imaging unit, wherein the tilted side camera platform/area is at an angle of about 2.0 to 3.0 degrees to said longitudinal axis of said secondary modular imaging unit.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 demonstrates a multi camera medical imaging device comprising a distal tip with at least two cameras, according to exemplary embodiments of the disclosed subject matter;

FIG. 2 demonstrates a distal tip of a multi camera medical imaging device comprising a primary modular imaging unit, a front modular imaging unit and a secondary modular imaging unit, according to exemplary embodiments of the disclosed subject matter;

FIG. 3A shows a perspective side view of a primary modular imaging unit comprising an optical gear module, according to exemplary embodiments of the disclosed subject matter;

FIG. 3B shows a perspective rear view of a primary modular imaging unit from the longitudinal opening or void, according to FIG. 3A;

FIG. 4 shows an exploded view of the primary modular imaging unit of the distal tip element of FIG. 1;

FIG. 5A shows a perspective front view of a front modular imaging unit of a distal tip designed to capture the distal tip's front field of view, according to exemplary embodiments of the disclosed subject matter;

FIG. 5B shows a perspective rear view of a front modular imaging unit of a distal tip designed to capture the distal tip's front field of view, according to FIG. 5A;

FIG. 6 shows an exploded view of a front modular imaging unit of the distal tip of FIG. 2;

FIGS. 7A-7C show a front illuminator electronic circuit and a side illuminator electronic circuit, designed to provide the light :source for the multi camera medical imaging device's camera, according to exemplary embodiments of the disclosed subject matter.

FIG. 8A shows a perspective side view of a secondary modular imaging unit functioning as a bracket which can be combined into a primary modular imaging unit, according to exemplary embodiments of the disclosed subject matter;

FIG. 8B shows a perspective rear view of a secondary modular imaging unit which can function as a bracket combined into a primary modular imaging unit, according to FIG. 8A;

FIG. 8C shows an exploded view of a secondary modular imaging unit, which can function as a bracket combined into a primary modular imaging unit, according to FIGS. 8A-8B;

FIG. 9A shows a first side perspective view of a distal tip of a multi camera medical imaging device comprising three imaging units, shown in an exploded view, according to exemplary embodiments of the disclosed subject matter;

FIG. 9B shows a second side perspective view of a distal tip, according FIG. 9A;

FIG. 10 shows a front and two side cameras located within the distal tip, where the front, primary and secondary modular imaging units shown in FIGS. 1 and 9A are removed;

FIG. 11 demonstrates a medical imaging device comprising at least one tilted camera and a changing diameter, according to another exemplary embodiment of the disclosed subject matter;

FIG. 12 shows a second side perspective view of a distal tip of a multi camera medical imaging device comprising three modular imaging units, according to exemplary embodiments of the disclosed subject matter;

FIG. 13A demonstrates a cross-sectional view of a distal tip of a multi camera medical imaging device with a tilted second lens assembly and sensor, according to FIG. 11;

FIG. 13B shows a cross-sectional view of a distal tip of a multi camera medical imaging device with a tilted secondary lens assembly and sensor, according to FIG. 13A;

FIG. 14A shows cross-sectional view of a multi camera medical imaging device with a tilted first lens assembly, according exemplary embodiments of the disclosed subject matter;

FIG. 14B shows cross-sectional view of a multi camera medical imaging device with a tilted first lens assembly and a tilted second lens assembly, according exemplary embodiments of the disclosed subject matter;

FIG. 15A shows a cross-sectional secondary modular imaging unit of a distal tip, according to exemplary embodiments of the disclosed subject matter;

FIG. 15B shows a schematic cross-sectional side view of a secondary modular imaging unit of a distal tip, according to FIG. 15A;

FIG. 16A shows a schematic view of a multi camera medical imaging device with a front camera and two side cameras and wherein each of the front and side cameras having lens assemblies creating a field of view of about 90 to 100 degrees, of about 95 degrees, according to exemplary embodiments of the disclosed subject matter;

FIG. 16B shows a schematic view of multi camera medical imaging device with a front camera comprising a front lens assembly with a field of view characterized with an opening angle of between 88 to 98 degrees and two side cameras comprising side lens assemblies each with field of views characterized with an opening angle of about 90 to 110 degrees, of about 104 degrees, according to exemplary embodiments of the disclosed subject matter, and;

FIG. 16C shows a schematic view of multi camera medical imaging device representing a number of conditional implementations of a front camera comprising a front lens assembly and two side cameras comprising side lens assembly, according to exemplary embodiments of the disclosed subject matter.

### DETAILED DESCRIPTION OF THE INVENTION

The subject matter in the present invention discloses a multi camera medical imaging device comprising a multi camera rigid endoscope designs to aid medical procedures such as inspection or surgery procedures within for example and not limited to the abdomen or pelvis through small incisions made into the body. Such multi camera medical imaging device may comprise two or more cameras designed to aid medical procedures such as inspection or surgery.

The term “optical gear module” or “optical gear” is used herein to depict a set of components that allow the multi camera medical imaging device to capture light and transform that light into at least two images. In some embodiments, lenses are employed to capture light, image capturing devices, such as sensors, are employed to transform that light into at least one image and illumination modules are employed to provide light. In some embodiments, a camera comprises a plurality of optics such as lens assembly and sensor and is configured to receive reflected light from target objects. In some embodiments, an optical gear located in a distal tip of the multi camera medical imaging device can comprise sensor and lenses (e.g., camera) and light sources required for the sensor functioning.

Image capturing devices may be Charged Coupled Devices (CCD's) or Complementary Metal Oxide Semiconductor (CMOS) image sensors, or other suitable devices comprising a light sensitive surface usable for capturing an image. In some embodiments, a sensor such as a Charge Coupled Device (CCD) or a Complementary Metal Oxide Semiconductor (CMOS) image sensor (for detecting the reflected light received by an optical element), is employed.

It should also be noted that a plurality of terms, as follows, appearing in this specification are used interchangeably to apply or refer to similar components and should in no way be construed as limiting: A “multi camera medical imaging rigid scope” may also be referred to as “endoscope”. A “camera” may also be referred to as an image capturing device/component, comprises a lens assembly and sensor. An “illuminator” may also be referred to as an “illumination source”. Illuminators may optionally be discrete illuminators and may include a light-emitting diode (LED), which may be a white light LED, an infrared light LED, a near infrared light LED, an ultraviolet light LED or any other LED and or combination of the same. A “niche” may also be referred to as a “shallow trough”.

It is noted that the term “endoscope” as mentioned to herein may refer particularly to a rigid scope, semi-rigid scope or flexible scope such as laparoscope, according to some embodiments of the disclosed subject matter, but is not limited only to laparoscope, and may include other applications such as industrial applications. The term “endoscope” may also refer to any instrument used to examine the interior of a hollow organ or cavity of the body.

As used in the specification, the term “working distance” is the ability of the lens assembly to maintain a desired amount of image quality as an object is positioned closer to and farther from best focus (e.g., depth of field). As used in the specification, the term “tilt” is used to describe an inclination of one plane to another. In some embodiments, an angle created between an imaginary line which continues a direction of view along a longitudinal axis of the medical device and an imaginary line which continues a direction of view which is not perpendicular.

In some embodiments of the disclosed subject matter, a dedicated structure of a distal tip can allow more than one camera to be directed simultaneously to one object in dissimilar angles. Such angle dissimilarities refer to at least two cameras positioned non-perpendicularly one to each other. The angle dissimilarities are used to show the same object at the same size in a panoramic and or surround image captured by multi cameras, as the multi cameras have diverse distances and angles to the object. In some cases, the dissimilarity in the cameras' angles to the objects may be achieved by a distal tip structured to allow directions of views which are non-perpendicularly one to each other. The angle dissimilarities resulted of the distal tip structure allow to combined a continues panoramic and or surround view formed of the diverse field of views captured by the cameras.

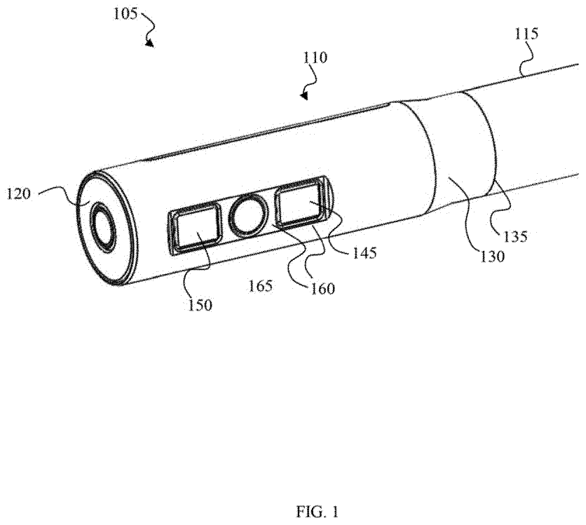

FIG. 1 demonstrates a multi camera medical imaging device comprising a distal tip having at least two cameras, according to exemplary embodiments of the disclosed subject matter. FIG. 1 shows a multi camera medical imaging device 105 comprising a distal tip 110 designed to be directly connected to a rigid shaft 115. The distal tip 110 may comprise an inclined surface 130 allowing to connect the distal tip 110 to the rigid shaft 115 provided in a narrower diameter than the diameter of the distal tip 110. The multi camera medical imaging device 105 also comprises a seamline 135 which outlines the connection line between the rigid shaft 115 and the inclined surface 130 of distal tip 110. In some cases, the rigid shaft 115 and the distal tip 110 may be connected by an adhesive material that seals the connection at the seamline 135. In some other cases, the rigid shaft 115 and the distal tip 110 may be connected by soldering. In possible embodiments of the disclosed subject matter, the rigid shaft 115 and the distal tip 110 may be connected by a screwing mechanism which fastens the rigid shaft 115 and the distal tip 110 together.

In some cases, the distal tip 110 may function as a multi camera section member designed to house at least two cameras. The distal tip 110 may comprise a front camera located at a front end of distal tip 110, denoted as a planar surface 120. Additional cameras may be located at the later round surface of the distal tip 110. In some embodiments, the front end of distal tip 110 has a tilted angle adapted to provide a tilted front surface. The cameras can be configured to aid medical and surgery procedures within for example and not limited to the abdomen or pelvis through small incisions made into the body. In some cases, such multi camera medical imaging device 105 can be utilized in laparoscopy wherein the multi camera medical imaging device can be inserted through a small incision in the body in order to perform medical procedures at the internal organs.

The distal tip 110 may also comprise a niche or a shallow trough 160 designed to house a first side camera optical window 165 and provide the opening required for the field of view of a first side camera (not shown). In some cases, the first side camera optical window 165 may comprise a transparent layer, such as glass or plastic, to isolate the first side camera (not shown) from liquids, gases and patient's debris and tissue. In some other cases, first side camera (not shown) may be covered by an optical window or more than one optical window. The niche 160 also enables emission of light from first side illuminator/s windows 150, and 145. In some cases, the light may be emitted by dedicated section illuminators such as light-emitting diodes, also known as LED. The dedicated section illuminators may be housed in windows 150, and/or 145. In some cases, the first side illuminator/s windows 145 and 150 may comprise a transparent layer, such as glass or plastic, to isolate the side illuminator/s from liquids, gases and patient's debris and tissue. In some other cases, first side illuminator/s windows 145 and 150 may comprise an optical window or more than one optical window. In yet some other embodiments, niche 160 may be covered by a transparent layer, such s glass or plastic or an optical window or more than one optical window for isolating the niche 160 from liquids, gases and patient's debris and tissue.

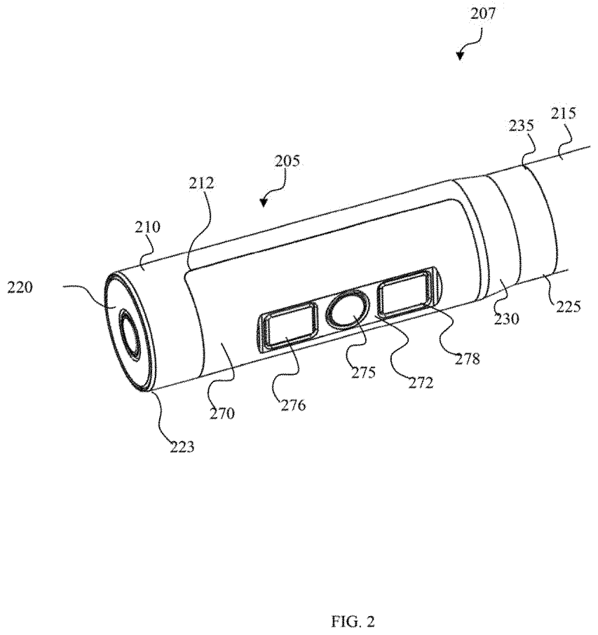

FIG. 2 demonstrates a distal tip of a multi camera medical imaging device comprising a primary modular imaging unit, a front modular imaging unit and a secondary modular imaging unit, according to exemplary embodiments of the disclosed subject matter. FIG. 2 shows a distal tip 205 of a multi camera medical imaging device 207, the distal tip 205 comprises a primary modular imaging unit 210 designed to be connected to a rigid shaft 215 of the multi camera medical imaging device 207. The primary modular imaging unit 210 has a longitudinal opening 212, the longitudinal opening 212 may be adapted to be aligned with a secondary modular imaging unit 270, such that secondary modular imaging unit 270 may function as a bracket enclosing the longitudinal opening 212 of primary modular imaging unit 210 along the length thereof. The distal tip 205 further comprises a front modular imaging unit 220 adapted to abut a distal front end 223 of the primary modular imaging unit 210. The three imaging units, primary modular imaging unit 210, front modular imaging unit 220 and secondary modular imaging unit 270, form the distal tip 205. In some cases, the secondary modular imaging unit 270 and the front modular imaging unit 220 may be attached to the primary modular imaging unit 210 in a sealed fashion, which prevents leaking of liquids, gases and/or debris and or body fluids into the distal tip 205. In some cases, the secondary modular imaging unit 270 and the front modular imaging unit 220 may be attached to the primary modular imaging unit 210 by an adhesive material, soldering, screwing mechanism and the like.

In some embodiments of the present invention, the secondary modular imaging unit 270 may be detachable from the primary modular imaging unit 210. In some cases, an optical gear located within the distal tip 205 may be mounted onto the secondary modular imaging unit 270, such that when detaching and pulling out the secondary modular imaging unit 270, the optical gear attached to the secondary modular imaging unit 270 may also be pulled out jointly with the modular secondary imaging unit 270.

The secondary modular imaging unit 20 may comprise a niche 272 designed to house a second side camera optical window 275 and provide the opening required for the field of view of a second side camera ( not shown). In some cases, the second side camera optical window 275 may comprise a transparent layer, such as glass or plastic, to isolate the second side camera (not shown) from liquids, gases and patient's debris and tissue. In some other cases, second side camera (not shown) may be covered by an optical window or more than one optical window. The niche 272 also enables emission of light from second side illuminator optical windows 276, and 278 for illuminating the area captured by the second side camera. In some cases, the light may be emitted by dedicated section illuminators such as light-emitting diodes, also known as LED. The dedicated section illuminators may be housed within the second side illuminator optical windows 276, and 278.

In some cases, the distal tip 205 may comprise an included surface 230 allowing to connect the distal tip 205 to the rigid shaft 215 provided in a narrower diameter than the diameter of the distal tip 205. The inclined surface 230 may ends at a rear end section 225 of the distal tip 205. The rear end section 225 of the distal tip 205 may be adapted to connect to the rigid shaft 215 at a seamline 235. In some cases, the rigid shaft 215 may be a semi-rigid shaft, or a flexible shaft which can be bent and manipulated by a user utilizing the multi camera medical imaging device 207.

In some embodiments of the present invention, the front modular imaging unit 220 may be detachable from the primary modular imaging unit 210. In some cases, an optical gear located within the distal tip 205 may be mounted onto the front modular imaging unit 220, such that when detaching and pulling out the front modular imaging unit 220, the optical gear attached to the front modular imaging unit 220 may also be pulled out jointly with the front modular imaging unit 220.

The three imaging units 210, 270 and 220 are designed to interlocked with each other and to fit over inner parts of the distal tip 205, and to provide protection to internal components within the inner part. The internal components within the inner parts of the distal tip 205 may comprise a lens assembly, a sensor, a foldable circuit board, an illuminator electronic circuit board, an illuminator module, and additional circuits and electrical components to convey and control the electrical signals and the electrical power required for the operation of the optical gears. The primary modular imaging unit 210, the secondary modular imaging unit 270 and the front modular imaging unit 220 are configured to abut to cover the distal tip 205 of the multi camera medical imaging device 207. In some cases, the three imaging units 210, 270 and 220 may be made of materials adapted to autoclave requirements such as but not limited to stainless steel.

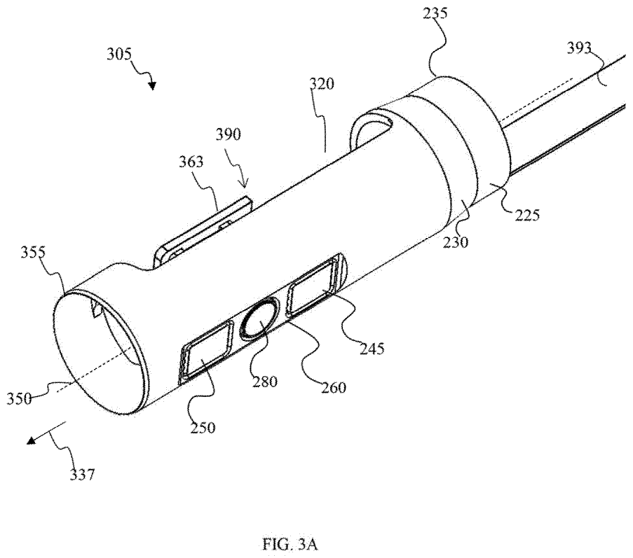

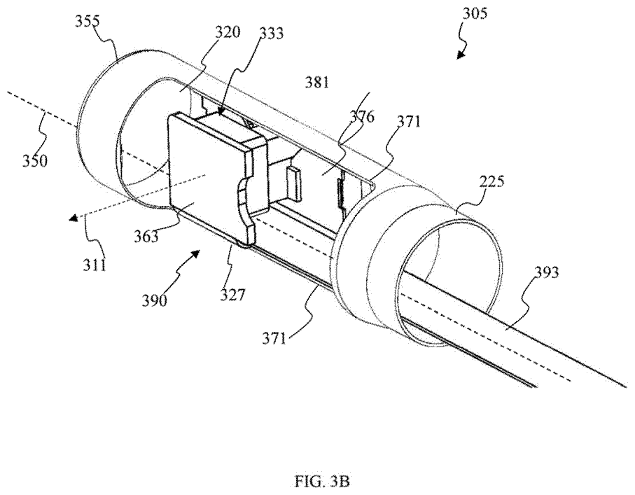

FIG. 3A shows a perspective side view of a primary modular imaging unit comprising an optical gear module, according to exemplary embodiments of the disclosed subject matter. FIG. 3A shows a primary modular imaging unit 305 which can be a part of a distal tip and connected to a rigid shaft (not shown), as aforementioned. In some cases, the primary modular imaging unit 305 may comprise an inclined surface 230 allowing to connect the primary modular imaging unit 305 to the rigid shaft provided in a narrower diameter than the diameter of the primary modular imaging unit 305. The inclined surface 230 may ends at a rear end section 225 of primary modular imaging unit 305. The rear end section 225 of primary modular imaging unit 305 may be adapted to connect to rigid shaft at a seamline 235. In other embodiments, the primary modular imaging unit 305 may comprise the same diameter as the rigid shaft (not shown) such that primary modular imaging unit 305 may be adapted to connect to rigid shaft at a seamline 235.

The primary modular imaging unit 305 may comprise a niche 260 designed to house a first side camera optical window 280 and provide the opening required for the field of view of a first side camera (not shown). In some cases, the first side camera optical window 280 may comprise a transparent layer, such as glass or plastic, to isolate the side camera from liquids, gases and patient's debris and tissue. In some other cases, first side camera optical window 280 may comprise an optical window or more than one optical window. The niche 260 also enables emission of light from first side illuminator optical window 245, and 250 for illuminating the area captured by the first side camera. In some cases, the light may be emitted by dedicated section illuminators such as light-emitting diodes, also known as LED. The dedicated section illuminators may be housed within the first side illuminator optical window 245 and 250.

The first side illuminator optical windows 245 and 250 adapted to cover first side illumination modules (not shown). The first side illumination modules situate such to provide illumination to the first side camera (not shown). The first side camera optical window 280 is adapted to cover the first side camera, wherein this first side camera typically comprises a sensor and a lens assembly (not shown). In some cases, the first side camera optical window 280 and the first side illuminator optical windows 245, and 250, may be made of materials adapted to autoclave requirements.

The primary modular imaging unit 305 further comprise a longitudinal opening 320 located on the primary modular imaging unit 305 lateral side and parallel to longitudinal axis 350 of primary modular imaging unit 305. The longitudinal opening 320 may be a void. The longitudinal opening 320 designed to provide the required freedom for fitting a first side optical gear located within the primary modular imaging unit 305. For example, in case the primary modular imaging unit 305 comprises a first side foldable circuit board 390 which may be designed to hold the first side optical gear required for imaging operation of a medical imaging device. In some embodiments, first side foldable circuit board 390 comprises a first side longitudinal circuit board 393 positioned in parallel to the primary modular imaging longitudinal axis 350 and extends outward from the rear end section 225 of primary modular imaging unit 305 into the rigid shaft (not shown). The first side foldable circuit board 390 further comprise a first side rigid circuit board 363 designed to carry the first side camera. The structure of the first side foldable circuit board 390 may allow locating the first side camera and first side illuminator circuit boards within the primary modular imaging unit 305. The primary modular imaging unit 305 may also comprise a distal front end 355 adapted to house a front modular imaging unit (not shown). Such, the front modular imaging unit may comprise a front optical gear required for capturing a front field of view.

FIG. 3B shows a perspective rear view of a primary modular imaging unit from the longitudinal opening or void, according to FIG. 3A. FIG. 3B shows a primary modular imaging unit 305 which can be a part of a distal tip and connected to a rigid shaft, such as distal tip 110 and rigid shaft 115 of FIG. 1. The primary modular imaging unit 305 comprises a longitudinal opening 320 shaped and designed to allow the required freedom for inserting, arranging, and in some cases, replacing a first side optical gear 333. The longitudinal opening 320 may be a void. The first side optical gear 333 can be mounted on a first side foldable circuit board 390 comprising a first side longitudinal circuit board 393 positioned in parallel to a primary modular imaging unit longitudinal axis 350 and extends outward from a rear end section 225 of primary modular imaging unit 305 into the rigid shaft (not shown). A foldable arm 327 may connect a first side rigid circuit board 363 to the first side longitudinal circuit board 393 of first side foldable circuit board 390. The first side rigid circuit board 363 is designed to carry a first side sensor (not shown) and a first side lens assembly 381. The structure of first side foldable circuit board 390 may allow locating the first side optical gear 333 within the primary modular imaging unit 305.

In some cases, the first side sensor may be configured to communicate the digital video signals to the first side longitudinal circuit board 393 via the first side rigid circuit board 363. The first side rigid circuit board 363 may be able to conduct the communications between the sensor and the first side lens assembly 381 to, and/or from the first side longitudinal circuit board 393. The conducted communications may be such s, digital video signals, electrical signals required for the operation of the first side optical gear 333, digitized data as a result of the first side optical gear 333 operation, and the like. In some cases, electrical power may also be conveyed via the first side longitudinal circuit board 393 to the first side optical gear 333.

In possible embodiments, the first side longitudinal circuit board 393 and the foldable arm 327 may be made of typical materials used for making circuit boards, such as ceramic, polyamides for flexible board, and glass-reinforced epoxy, and the like such, also provide the elastic movement required for the rotations of the foldable arm 327 along axis 311. In some cases, the foldable arm 327 may comprise hinges which can provide the ability to rotate/bend the foldable arm 327 upwardly or downwardly along axis 311. The foldable arm 327 can be situated horizontally to the first side longitudinal circuit board 393 or, be bended upwardly, essentially vertically to longitudinal axis 350. For example, the foldable arm 327 can bend around and be positioned between 45 to 95 degrees to the longitudinal axis 350. In some cases, the first side rigid circuit board 363 can bend further with the foldable arm 327 and be positioned essentially vertically to the first side longitudinal circuit board 393, with about 90 degrees between the foldable arm 327 and the longitudinal axis 350. In some cases, the foldable arm 327 may be situated with any angle on the range between 0 and 95 degrees to the longitudinal axis 350 of primary modular imaging unit 305. The foldable arm 327 can be adapted to bend the first side rigid circuit board 363 to a range of angles design to capture and provide the primary modular imaging unit 305 with a first side field of required for the operation of the medical imaging rigid scope.

The primary modular imaging unit 305 also comprises a first side illuminator electronic circuit board 376 designed to host the illumination modules (not shown) which provides the required light source for the operation of the first side camera. The first side illuminator electronic circuit board 376 may comprise additional circuits and electrical components to convey and control the electrical signals and the electrical power required for the operation of the illumination modules. Thus, in some cases, the first side illuminator electronic circuit board 376 may be connected to the first side foldable circuit board 390 for receiving the electrical power required for the operation and for controlling the light intensity of the illumination modules.

In some embodiments of the disclosed subject matter, the primary modular imaging unit 305 may comprise a detachable fitting element/s 371 surrounding the edge of longitudinal opening 320. The detachable fitting element/s 371 can be fastened to matching fitting element/s of a secondary modular imaging unit (not shown). In some cases, detachable fitting element/s 371 may be fastened, glued, or soldered to the fitting element/s of the secondary modular imaging unit (not shown). The detachable fitting element/s 371 are for example and not limited to protruding part, fit holding rib, fit holding groove and the like. The detachable fitting element/s 371 is configured to detachably fitted within the secondary modular imaging unit which result in a sealed closing between the two modular imaging units that essentially prevent entry of debris, fluids and or gases to inner parts of the isolated two modular imaging units.

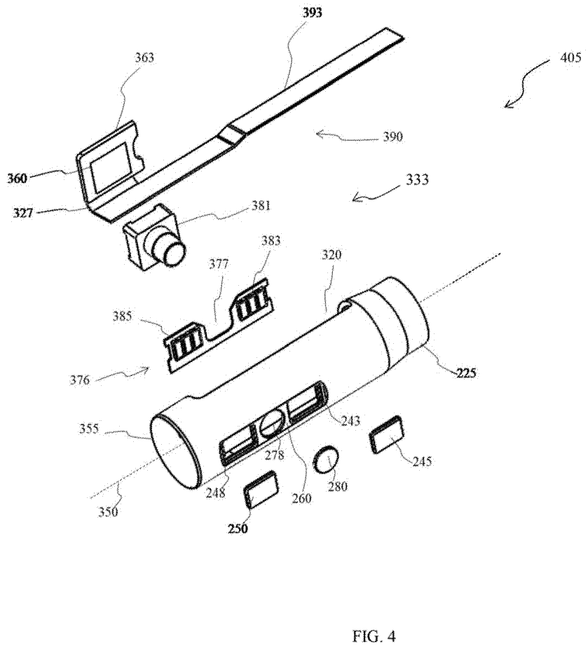

FIG. 4 shows exploded view of a primary modular imaging unit of the distal tip according to FIG. 1. FIG. 4 shows a primary modular imaging unit 405 designed to employ a distal tip's equipment required for the operations thereof, for example distal tip 110 of a multi camera medical imaging device 105 as described with reference to FIG. 1. The primary modular imaging unit 405 comprises a niche 260 designed to house a first side camera optical window 280. The first side camera optical window 280 can be adapted to cover a first side lens assembly 381 and be inserted within a first side camera aperture 278. The first side camera aperture 278 is configured to hold, support and secure first side lens assembly, within the lateral surface of primary modular imaging unit 405. The first side camera optical window 280 is adapted to provide the opening required for the field of view of the first side lens assembly 381. In some cases, the first side camera optical window 280 may comprise a transparent layer, such as glass or plastic, to isolate the first side lens assembly 381 from liquids, gases and patient's debris and tissue. In some cases, the first side camera optical window 280 may comprise semi-transparent layer allowing a part of the light spectrum to pass out from the first side camera optical window 280. In some cases, the first side camera optical window 280 may comprise a light filter to block a portion of the light spectrum from pass to the first side lens assembly 381 and o a first side sensor 360.

The primary modular imaging unit 405 further comprises a first side illuminator optical window 245 which can be inserted into a first side illuminator opening 243, and a first side illuminator optical window 250 which can be inserted into a first side illuminator opening 248. The first side camera aperture 278 can be located between first side illuminator opening 243 and first side illuminator opening 248 within the primary modular imaging unit 405 lateral surface. Typically, first side camera aperture 278 and first side illuminator openings 243 and 248 are aligned along a longitudinal axis of the primary modular imaging unit 405 within niche 260.

In some embodiments, the first side illuminator optical windows 245 and 250 may comprise a transparent layer, such as glass or plastic, to isolate the first side illuminator/s from liquids, gases and patient's debris and tissue. In some other embodiments, first side illuminator optical windows 245 and 250 may comprise an optical window or more than one optical window. The first side illuminator optical windows 245 and 250 enable emission of light emitted by first side illumination modules 383 and 385, respectively

Such, a first side camera comprises the first side lens assembly 381 and the first side sensor 360 may provide a field of views between 80 to 130 degrees and a working distance in a range of 1-30 millimeters, in a range of 5-150 millimeters, wherein the first side illumination modules 383 and 385 adapted to illuminate such field of view and working distance.

In some embodiments, the first side illuminator optical windows 245 and 250, and the first side camera optical window 280 may be connected to the first side illuminator openings 243 and 248 and to first side camera aperture 278, respectively, by adhesive material, screws, soldering, clamping devices, and the like. In some embodiments, first side illuminator optical windows 245 and 250, and the first side camera optical window 280 may be attached to first side illuminator openings 243 and 248 and to first side camera aperture 278, respectively in replaceable manner techniques, to enable the replacement of first side illuminator module 383 and or first side illuminator module 385 and or any illuminator within these modules and some of the lenses of the first side lens assembly 381.

The primary modular imaging unit 405 may also comprise a longitudinal opening 320 shaped and designed to allow the required freedom for accepting the first side optical gear located within the primary modular imaging unit 405. The longitudinal opening 320 may be a void. In some cases, the longitudinal opening 320 may be utilized to assemble the first side optical gear and the electrical equipment within the primary modular imaging unit 405. The primary modular imaging unit 405 further comprises a first side illuminator electronic circuit board 376, the first side illuminator electronic circuit board 376 holds first side illumination modules 383, 385 in place. The first side illuminator electronic circuit board 376 has a “U” opening 377 shaped to accommodate the first side lens assembly 381. Thus, the first side illuminator electronic circuit board 376 can be positioned within the primary modular imaging unit 405 such that the “U” opening 377 is adapted to aligned with the first side camera aperture 278 of the primary modular imaging unit 405. The first side illuminator openings 243 and 248 are configured to hold, support and secure the first side illuminator electronic circuit board 376 along with the first side illumination modules 383 and 385, within the lateral surface of primary modular imaging unit 405.

The first side lens assembly 381 may comprise a set of lenses employed to capture light and transmit the captured light to the first side sensor 360. The first side lens assembly 381 may capture and determine a first side field of view, which can be the field of view captured by the primary modular imaging unit 405. Optionally, the set of lenses of the first side lens assembly 381 and first side sensor 360 are configured to have a field of view of at least 80 degrees and up to essentially 135 degrees, and a working distance of at least 1 millimeters and up to essentially 150 millimeters.

The first side lens assembly 381 may connect to the first side sensor 360 for inclusion with a first side foldable circuit board 390. In some embodiments of the disclosed subject matter, the first side foldable circuit board 390 comprises a first side longitudinal circuit board 393 positioned in parallel to a primary modular imaging unit longitudinal axis 350. In some cases, the first side longitudinal circuit board 393 can also extend outward from the primary modular imaging unit 305 into the rigid shaft (not shown). The first side foldable circuit board 390 further comprise a first side rigid circuit board 363 designed to carry the first side sensor 360 and the first side lens assembly 381. The first side longitudinal circuit board 393 can be positioned in parallel to the primary modular imaging unit longitudinal axis 350 and extend outward from a rear end section 225 of primary modular imaging unit 405 into the rigid shaft (not shown). A foldable arm 327 may be connected the first side rigid circuit board 363 to the first side longitudinal circuit board 393. In some cases, the foldable arm 327 may be situated with any angle on the range between 0 and 95 degrees to the longitudinal axis 350 of primary modular imaging unit 405, such that first side rigid circuit board 363 may be situated with any angle on the range between 0 and 95 degrees to the longitudinal axis 350 of primary modular imaging unit 405. The structure of first side foldable circuit boar 390 allows placing, holding and operating the first side optical gear and such, provides the required field of view for operating a multi camera medical imaging device.

The primary modular imaging unit 405 may also comprise a distal front end 355 adapted to hold and support a front modular imaging unit (not shown). Such the front modular imaging unit may comprise the optical gear required for capturing a field of view at the front of the primary modular imaging unit 405.

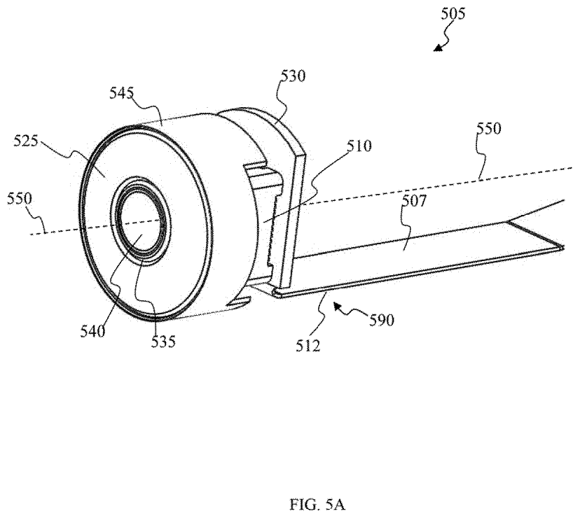

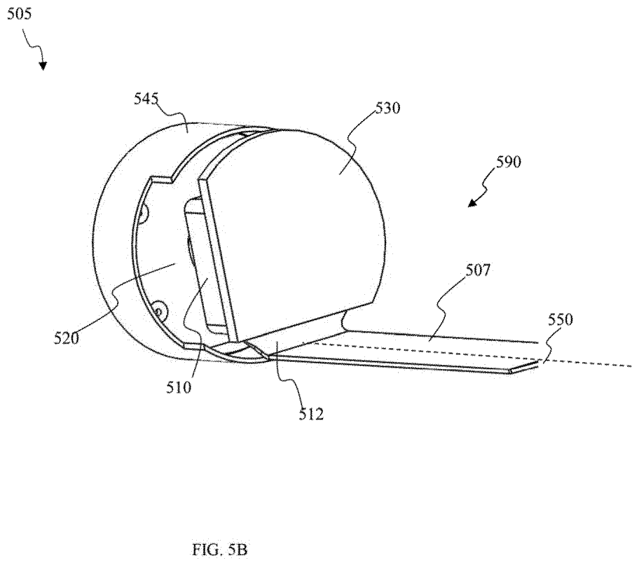

FIG. 5A shows a perspective front view of a front modular imaging unit of a distal tip designed to capture front field of view of a multi camera medical imaging device, according to exemplary embodiments of the disclosed subject matter. FIG. 5A shows a front modular imaging unit 505 which can be inserted into a distal front end of a primary modular imaging unit, for example distal front end 223 and distal front end 355 of primary modular imaging unit 305 as described with reference to FIGS. 2 and 3-4, respectively. The front modular imaging unit 505 comprises a front foldable circuit board 590, the front foldable circuit board 590 may comprise a front rigid circuit board 530 connected to a front longitudinal circuit board 507 via a front foldable arm 512, such that the front rigid circuit board 530 is situated essentially in a vertical position to the front longitudinal circuit board 507. The front longitudinal circuit board 507 may be positioned in parallel to a longitudinal axis 550 and extends outward from the primary modular imaging unit (not shown) into a rigid shaft (not shown) of a multi camera medical imaging device as described with reference to FIG. 2. In some cases, the front foldable arm 512 may be bent such that a horizontal angle is formed between the front rigid circuit board 530 and the front longitudinal circuit board 507. In some embodiments, the front foldable arm 512 can bend to position the front rigid circuit board 530 between 45 to 95 degrees to the longitudinal axis 550. In some cases, the front rigid circuit board 530 can bend further and be positioned essentially vertically to the front longitudinal circuit board 507, with about 90 degrees between the front foldable arm 512 and the longitudinal axis 550. In some cases, the front foldable arm 512 may be situated with any angle on the range between 0 and 95 degrees to the longitudinal axis 550. In some embodiments of the disclosed subject matter, the front foldable arm 512 may comprise hinges which can provide the ability to rotate/bend outwardly and/or inwardly.

In some embodiments of the disclosed subject matter, a front sensor (not shown) may be attached to the front rigid circuit board 530. In some cases, the front sensor may be configured to communicate the digital video signals to the front foldable circuit board 590 via the front rigid circuit board 530. The front rigid circuit board 530 may be able to convey the communications between the front sensor and a front lens assembly 510 located within a front frame 545 to or from the front foldable circuit board 590. The conveyed communication may be such as, digital video signals, electrical signals and the like, required for the operation of the distal tip of the multi camera medical imaging device. In some cases, electrical power may also be conveyed via the front foldable circuit board 590 to front rigid circuit board 530.

The front modular imaging unit 505 further comprises a front illuminator optical window 525 comprising a transparent material such as glass or plastic designed to allow the light to enlighten through the optical window, such, the light emitted by illumination modules (not shown) is spread out to the outer space of the front modular imaging unit 505. The front illuminator optical window 525 may be attached to the front frame 545. In some cases, the front frame 545 may be a round shaped element. In some other cases, the front frame 545 may be an oval shaped frame, or other shapes, according to some medical procedure's requirements. The front illuminator optical window 525 may be attached to the front frame 545 in a sealed fashion preventing leaks of liquids and/or body fluids into the front modular imaging unit 505. In some cases, the front illuminator optical window 525 may be attached to the front frame 545 by an adhesive material, screws, soldering, clamping devices, and the like. In some embodiments, the front illuminator optical window 525 may be attached to front frame 545 in detachable manner techniques, this enables the replacement of front illuminator modules (not shown) or any illuminator within these modules. The front illuminator optical window 525 may comprise an aperture 535 adapted to house a front lens assembly window 540. The front lens assembly window 540 comprises a transparent material such as glass or plastic in order to allow the light to pass through the optical window and be captured by the front sensor (not shown). The front rigid circuit board 530 designed to carry front sensor (not shown) and the front lens assembly 510. The structure of the front foldable circuit board 590 may allow locating the front sensor (not shown) and the front lens assembly 510 within the front modular imaging unit 505 such that the front lens assembly 510 can be aligned with the aperture 535 and the front lens assembly window 540. In some cases, the front illuminator optical window 525 and the front lens assembly window 540 may be made of materials adapted to autoclave requirements.

FIG. 5B shows a perspective rear view of a front modular imaging unit of a distal tip designed to capture the distal tip's front field of view, according to FIG. 5A. FIG. 5B shows a front modular imaging unit 505 which can be inserted into a distal front end of a primary modular imaging unit, for example distal front end 355 of primary modular imaging unit 305 as described with reference to FIGS. 3-4. The front modular imaging unit 505 comprises a front frame 545 which can function as a cover element for the equipment located at the distal tip, such as distal tip 205 of a multi camera medical imaging device 207 as described with reference to FIG. 2. The front frame 545 may also be inserted into the primary modular imaging unit (not shown). The front modular imaging unit 505 further comprises a front foldable circuit board 590, the front foldable circuit board 590 comprise a front rigid circuit board 530 connected to a front longitudinal circuit board 507 via a front foldable arm 512. The front longitudinal circuit board 507 may be positioned in parallel to a longitudinal axis 550.

The front foldable arm 512 can bend around to be positioned between 45 to 95 degrees to the longitudinal axis 550. In some cases, the front rigid circuit board 530 can bend further with the foldable arm 327 and be positioned essentially vertically to the front longitudinal circuit board 507. In some cases, the front foldable arm 512 may be situated with any angle on the range between 0 and 95 degrees to the longitudinal axis 550 of front modular imaging unit 505.

The front rigid circuit board 530 is adapted to convey the communications between the front sensor (not shown) and the front lens assembly 510 located within the front frame 545 to, or from the front foldable circuit board 590. The front rigid circuit board 530 may also be attached to a front illuminator electronic circuit board 520, designed to house and operate the illumination modules (not shown) employed to provide the light source for the front lens assembly 510 and front sensor (not shown) of the multi camera medical imaging device.

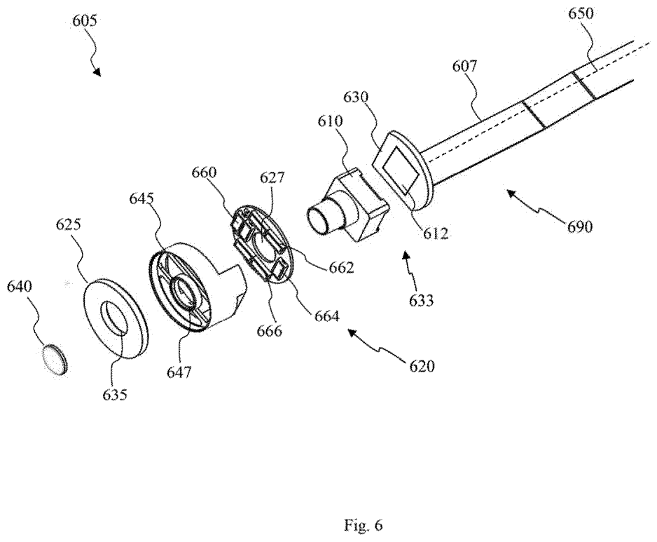

FIG. 6 shows an exploded view of a front modular imaging unit of the distal tip of FIG. 2. FIG. 6 shows a front modular imaging unit 605 designed to employ a distal tip's equipment required for the operations thereof, for example distal tip 205 of a multi camera medical imaging device 207 as described with reference to FIG. 2. The front modular imaging unit 605 comprises a front frame 645 designed to house a front camera optical window 640 and a front illuminator optical window 625. The front camera optical window 640 is adapted to cover a front lens assembly 610 and to be inserted within a front frame camera aperture 647. The front frame camera aperture 647 configured to hold, support and secure front lens assembly 610 within the front modular imaging unit 605. In some embodiments, the front frame camera aperture 647 may situated at the center of the front frame 645. In some embodiments of the disclosed subject matter, the front frame camera aperture 647 may situated close to the front frame 645 edge. The front camera optical window 640 is adapted to provide the opening required for the field of view of the front lens assembly. In some cases, the front camera optical window 640 may comprise a transparent layer, such as glass or plastic, adapted to isolate the front lens assembly 610 from liquids, gases and patient's debris and tissue. In some cases, the front camera optical window 640 may comprise semi-transparent layer allowing a part of the light spectrum to pass in from the front camera optical window 640. In some cases, the front camera optical window 640 may comprise a light filter to block a portion of the light spectrum from pass to the front lens assembly 610 and to a front sensor 612.

The front modular imaging unit 605 further comprises a front illuminator optical window 625 which can be inserted into the front frame 645. The front illuminator optical window 625 may comprise an aperture 635 adapted to house the front camera optical window 640. The front illuminator optical window 625 is shaped to fit within front frame 645 such that aperture 635 is aligned with the front frame camera aperture 647.

In some embodiments of the disclosed subject matter, the front illuminator optical window 625 may comprise a transparent layer, such as glass or plastic, to isolate front illuminator/s from liquids, gases and patient's debris and tissue. In some other embodiments, front illuminator optical window 625 may comprise an optical window or more than one optical window. The front illuminator optical window 625 enable emission of light emitted by front illumination modules such as front illumination modules 660, 662, 664 and 666. A front camera comprises the front lens assembly 610 and the front sensor 612 may provide a field of view between 80 to 130 degrees and a working distance in a range of 1-30 millimeters, in a range of 5-150 millimeters, wherein the front illumination modules 660, 662, 664 and 666 adapted to illuminate the front camera field of view and working distance of the front camera.

In some embodiments of the disclosed subject matter, the front illuminator optical window 625 may be connected to the front frame 645 by adhesive material, screws, soldering, clamping devices, and the like. In some embodiments, the front illuminator optical window 625 may be attached to front frame 645 in detachable manner techniques, this enables the replacement of front illumination modules 660, 662, 664 and 666 or any illuminator within these modules.

In some embodiments of the disclosed subject matter, the front camera optical window 640 may be connected to the front frame camera aperture 647 by adhesive material, screws, soldering, clamping devices, and the like. In some embodiments, the front camera optical window 640 may be attached to front frame camera aperture 647 in detachable manner techniques, this enables the replacement of some of the lenses of the front lens assembly 610.

In other embodiments of the disclosed subject matter, the front camera optical window 640 may be connected to the front illuminator optical window 625 by adhesive material, screws, soldering, clamping devices, and the like. In some embodiments, the front camera optical window 640 may be attached to front illuminator optical window 625 in detachable manner techniques, this enables the replacement of some of the lenses of the front lens assembly 610.

The front modular imaging unit 605 further comprise a front illuminator electronic circuit board 620 adapted to hold, support and secure the front illumination modules 660, 662, 664 and 666 within the front modular imaging unit 605. In other possible embodiments of the disclosed subject matter, the number and location of front illumination modules 660, 662, 664 and 666 may vary for example, less than 4 front illumination modules or more, wherein the front illumination module may hold 1, 2, 3, 4 or more LED. In some cases, said LED's may emit light at the same light spectrum. In cases, said LED's may emit light at different light spectrum.

The front illuminator electronic circuit board 620 has a front illuminator electronic circuit board aperture 627 shaped to accommodate the front lens assembly 610. Thus, the front illuminator electronic circuit board 620 can be positioned within the front modular imaging unit 605 such that the front illuminator electronic circuit board aperture 627 is adapted to be aligned with the front frame camera aperture 647 and aperture 635 of front illuminator optical window 625 to provide the openings required for the field of view of the front lens assembly 610 and front sensor 612, e.g., a front camera 633.

The front lens assembly 610 may comprise a set of lenses employed to capture light and transmit the captured light to the front sensor 612. The front lens assembly 610 may determine a field of view, which can be the field of view captured by the front modular imaging unit 605. Optionally, the set of lenses of the front lens assembly 610 is configured to have a field of view of at least 80 degrees and up to essentially 135 degrees and a working distance of at least 1 millimeters and up to essentially 150 millimeters.

The front lens assembly 610 connects to the front sensor 612 for inclusion with a front foldable circuit board 690. In some embodiments, front foldable circuit board 690 may comprise a front rigid circuit board 630 connected to a front longitudinal circuit board 607 via a front foldable arm (not shown), such that the front rigid circuit board 630 is situated essentially in a vertical position to the front longitudinal circuit board 607. The front longitudinal circuit board 607 may be positioned in parallel to a longitudinal axis 650 and extends outward from a primary modular imaging unit (not shown) into a rigid shaft (not shown) of a multi camera medical imaging device as described with reference to FIG. 2. In some cases, the foldable arm (not shown) may be situated with any angle on the range between 0 and 95 degrees to the longitudinal axis 650.

In some embodiments, the front sensor 612 may be attached to the front rigid circuit board 630. In some embodiments, the front sensor 612 may be configured to communicate the digital video signals to the front foldable circuit board 690. The front rigid circuit board 630 may be able to convey the communications between the front camera 633 located within a front frame 645 to or from the front foldable circuit board 690. The conveyed communication may be such as, digital video signals, electrical signals and the like required for the operation of the distal tip of the multi camera medical imaging device.

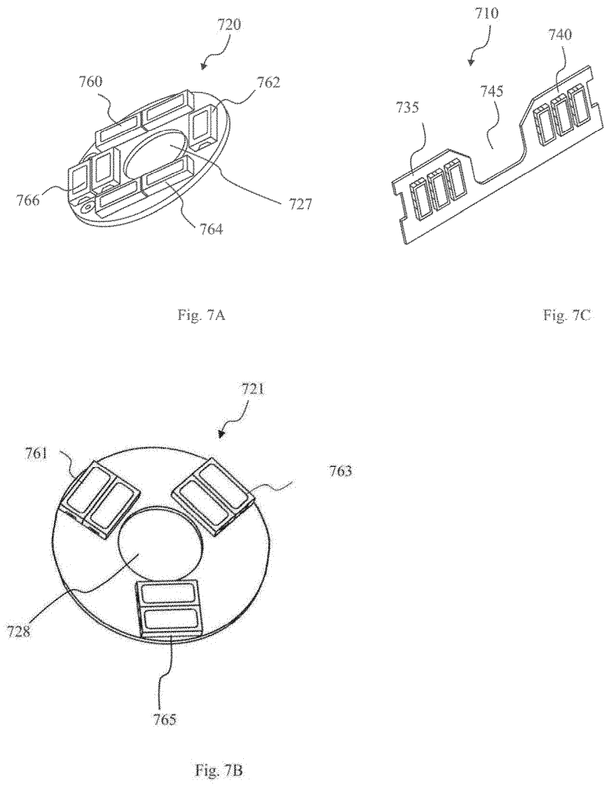

FIGS. 7A-7C show a front illuminator electronic circuit board and a side illuminator electronic circuit board, designed to provide the light source for the multi camera medical imaging device's camera, according to exemplary embodiments of the disclosed subject matter. FIG. 7A shows a front illuminator electronic circuit board 720 which in some embodiments of the disclosed subject matter, can hold a set of four front illumination modules, represented as front illumination modules 760, 762, 764, and 766, and be adapted to secure and accommodate a front camera (not shown) within a front illuminator electronic circuit board aperture 727. In some cases, the front illuminator electronic circuit board aperture 727 may not be positioned in the center of the front illuminator electronic circuit board 720. The front illumination modules 760, 762, 764, and 766 may hold a different number of illuminators. In some embodiments, front illuminator electronic circuit board 720 can hold a set of 1, 2, 3, 4, 5 or more front illumination modules. In other possible embodiments of the disclosed subject matter, the number and location of front illuminators modules may vary for example, less than 4 front illumination modules or more, wherein each front illumination modules may hold 1, 2, 3, 4 or more LEDs. In some cases, said. LED's may emit light at the same light spectrum. In some cases, said LED's may emit light at different light spectrum. The front illumination modules are adapted to provide the light source required for the front sensor functioning, and also enable emission of light for illuminating the area captured by the front camera.

In some embodiments of the disclosed subject matter, the front illuminator electronic circuit board aperture 727 may be positioned in the center of the front illuminator electronic circuit board 721 as shown in FIG. 7B. A front illuminator electronic circuit board such as front illuminator electronic circuit board 721 may comprise a front central illuminator electronic circuit board aperture 728 positioned at the center of the front illuminator electronic circuit board 721. In some embodiments of the disclosed subject matter, the front illuminator electronic circuit board 721 may be adapted to hold a set of three front illumination modules, represented as front illumination modules 761, 763, and 765. The front illuminator electronic circuit board 721 may also be adapted to secure and accommodate a front camera (not shown) within the front central illuminator electronic circuit board aperture 728. In some embodiments of the disclosed subject matter, the front illuminator electronic circuit board 721 can hold a set of 1, 2, 3, 4, 5 or more front illumination modules. In other possible embodiments of the disclosed subject matter, the number and location of front illuminators modules may vary. For example, less than 3 front illumination modules or more, wherein the front illumination modules may hold 1, 2, 3, 4 or more LEDs. In some cases, said LED's may emit light at the same light spectrum. In some cases, said LED's may emit light at different light spectrum

FIG. 7C shows a side illuminator electronic circuit board 710 designed to support and host side illumination modules which provide the required light source to a lateral surface of a multi camera medical imaging device. The side illuminator electronic circuit board 710 holds a side illumination module 735 and side illumination module 740 in place. In some embodiments of the disclosed subject matter, the side illumination modules 735 and 740 may hold 1 or more illuminators such as LED. In some cases, said LED's may emit light at the same light spectrum. In some cases, said LED's may emit light at different light spectrum In some cases, the module may hold a different number of illuminators such as LED. In some cases, said LED's may emit light at the same light spectrum. In some cases, said LED's may emit light at different light spectrum. In some embodiments of the disclosed subject matter, the side illuminator electronic circuit board 710 has a U-shaped opening 745 shaped to accommodate a side camera (not shown). The side illumination modules 735 and 740 may be on either side of the side camera or at one side of the side camera enable emission of light for illuminating the area captured by the side camera.

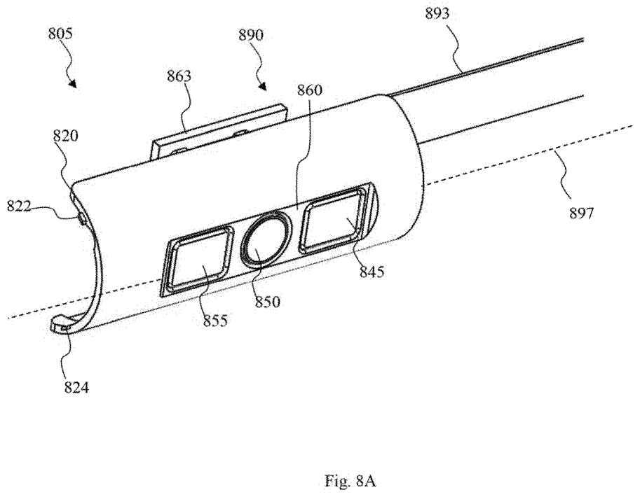

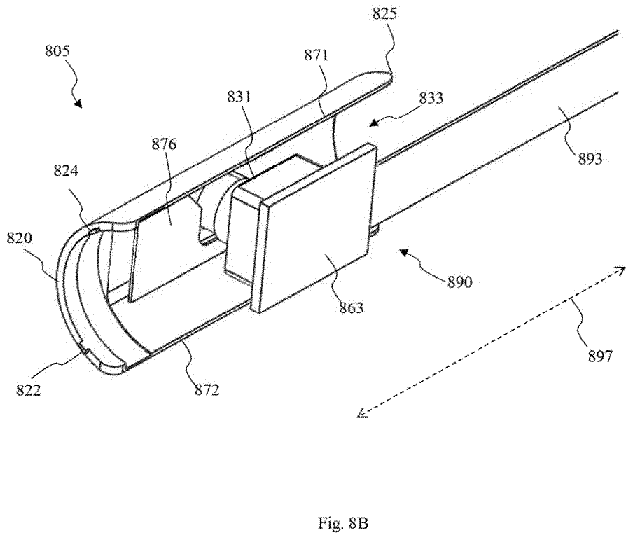

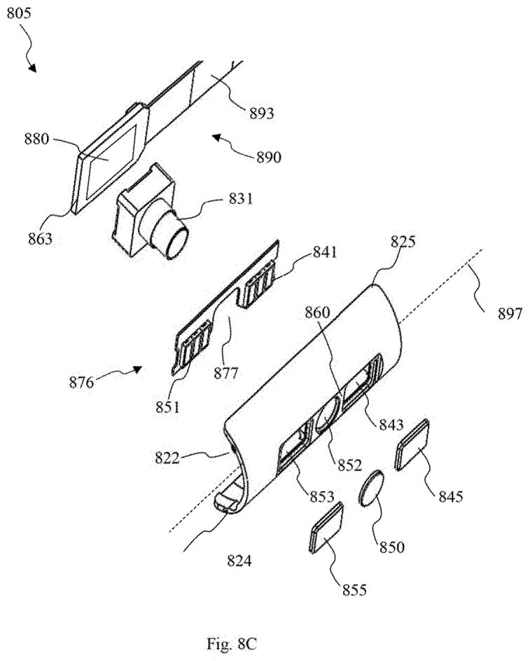

FIG. 8A shows a perspective side view of a secondary modular imaging unit functioning as a bracket which can be combined into a primary modular imaging unit, according to exemplary embodiments of the disclosed subject matter. FIG. 8A shows a secondary modular imaging unit 805 adapted to be interlocked with a primary modular imaging unit (not shown) and a front modular imaging unit (not shown) to employ a distal tip's equipment required for the operations thereof, for example distal tip 205 of a multi camera medical imaging device 207 as described with reference to FIG. 2. In some embodiments of the disclosed subject matter the secondary modular imaging unit 805 may comprise an optical gear configured to capture digital images and/or digital video images captured by the optical gear connected to the secondary modular imaging unit 805,

The secondary modular imaging unit 805 may comprise a U-shape haring a second side niche 860 located on a lateral side parallel to longitudinal axis 897 of secondary modular imaging unit 805. The second side niche 860 is designed to house a second side camera optical window 850 and provide the opening required for the field of view of a second side camera (not shown). In some cases, the second side camera optical window 850 may comprise a transparent layer, such as glass or plastic, to isolate the side camera from liquids, gases and patient's debris and tissue. In some other cases, the second side camera optical window 850 may comprise an optical window or more than one optical window. The second side niche 860 also enables emission of light from second side illuminator optical windows 845 and 855 for illuminating the area captured by the second side camera. In some cases, the light may be emitted by dedicated section illuminators (not shown) such as light-emitting diodes, also known as LED. The dedicated section illuminators may be housed within the second side illuminator optical windows 845 and 855.

The second side niche 860 of the secondary modular imaging unit 805 can house the second side illuminator optical windows 845 and 855 adapted to cover second side illumination modules (not shown). The second side illumination modules situate such to provide illumination to the second side camera (not shown). The second side niche 860 may also house the second side camera optical window 850, the second side camera optical window 850 is adapted to cover second side camera, wherein this camera typically comprises a sensor and a lens assembly (not shown).

The U-shape of secondary modular imaging unit 805 may adapted to provide the required freedom for fitting the second side optical gear located within the secondary modular imaging unit 805. For example, in case the secondary modular imaging unit 805 comprises a second side foldable circuit board 890 designed to hold the optical gear required for imaging operation of a multi camera medical imaging device. In same embodiments, second side foldable circuit board 890 comprises a second side longitudinal circuit board 893 positioned in parallel to the secondary modular imaging unit longitudinal axis 897 and extends outward from the secondary modular imaging unit 805 into the primary modular imaging unit and into a rigid shaft (both not shown). The second side foldable circuit board 890 further comprise a second side rigid circuit board 863 designed to carry a second side optical gear. The structure of the second side foldable circuit board 890 may allow locating the second side camera and side illuminator circuit boards within the secondary modular imaging unit 805. The secondary modular imaging unit 805 further comprise an edge 820 of its shape adapted to interlocked with the primary modular imaging unit (not shown).

The secondary modular imaging unit 805 may further comprise one or more protruding element located at the margins of edge 820 of the secondary modular imaging unit 805 to be inserted into corresponding openings in the primary modular imaging unit (not shown of the distal tip as discussed with reference to FIGS. 2-4, for example, protruding elements such as protruding elements 822 and 824. The protruding elements 822 and 824 may configured to fit and fasten the secondary modular imaging unit 805 to the primary modular imaging unit (not shown), results a sealed closing between the two modular imaging units that essentially prevent entry of debris, fluids and or gases to inner parts of the isolated two modular imaging units. In some cases, the two protruding elements 822 and 824 may be inserted into corresponding slots or grooves located within longitudinal opening or void of the primary modular imaging unit. In such cases, closing the distal tip by attaching the secondary modular imaging unit 805 and inserting the protruding elements 822 and 824 into the corresponding elements such as slots or grooves to provide a sealed, firmed and steady distal tip. In other embodiments, protruding elements 822 and 824 are slots or grooves adapted to protruding elements within the primary modular imaging unit. In some embodiments, protruding elements 822 and 824 of secondary modular imaging unit 805 configure to detachably fitted within detachable fitting elements of the primary modular imaging unit. For example, detachable fitting element/s 371 of primary modular imaging unit 305 as described with reference to FIG. 3B. In some embodiments, the number and the location of the protruding elements may vary from 1 to 10 or more along the margins of edge 820. In other embodiments, the secondary modular imaging unit 805 may fit into longitudinal opening or void of the primary modular imaging unit without any protruding elements. FIG. 8B shows a perspective rear view of a secondary modular imaging unit which can function as a bracket combined into a primary modular imaging unit, according to FIG. 8A. FIG. 8B shows a secondary modular imaging unit 805 designed to fit into a longitudinal opening or void of a primary modular imaging unit to create a cylinder or a cylinder like geometric figure adapted to a shape of a distal tip as described with reference to FIG. 2. In some cases, the secondary modular imaging unit 805 can function as a lid and thereby seal the primary modular imaging unit (not shown).

The secondary modular imaging unit 805 comprises a U-shape designed to allow the required freedom for inserting, arranging, and in some cases, replacing a second side optical gear 833. A second side optical gear 833 can be mounted on a second side foldable circuit board 890 comprising a second side longitudinal circuit board 893 positioned in parallel to the secondary modular imaging unit longitudinal axis 897 and extends outward from a rear end section 825 of secondary modular imaging unit 805 into the primary modular imaging unit (not shown). The second side rigid circuit board 863 connected to the second side longitudinal circuit board 893 and designed to carry a second side sensor (not shown) and a second side lens assembly 831. The structure of second side foldable circuit board 890 may allow locating the second side optical gear 833 within the secondary modular imaging unit 805.

In some embodiments, the second side sensor may configure to communicate the digital video signals to the second side longitudinal circuit board 893 via the second side rigid circuit board 863. The second side rigid circuit board 863 may be able to conduct the communications between the second side sensor and the second side lens assembly 831 to, and/or from the second side longitudinal circuit board 893. The conducted communications may be such as, digital video signals, electrical signals required for the operation of the second side optical gear 833, digitized data as a result of the second side optical gear 833 operation, and the like. In some cases, electrical power may also be conveyed via the second side longitudinal circuit board 893 to the second side optical gear 833.

In possible embodiments, the second side longitudinal circuit board 893 may be made of typical materials used for making circuit boards, such as ceramic, polyamides for flexible board, and glass-reinforced epoxy, and the like such, also provide the elastic movement of the second side longitudinal circuit board 893 along longitudinal axis 897. The second side rigid circuit board 863 can be situated parallel to the second side longitudinal circuit board 893, essentially horizontally to longitudinal axis 897 of secondary modular imaging unit 805.

The secondary modular imaging unit 805 also comprises a second side illuminator electronic circuit board 876 designed to host illumination modules (not shown) which provide the required light source to a second side lens assembly 831 and a second side sensor (not shown). The second side illuminator electronic circuit board 876 may comprise additional circuits and electrical components to convey and control the electrical signals and the electrical power required for the operation of the illumination modules. Thus, in some cases, the second side illuminator electronic circuit board 876 may be connected to the second side foldable circuit board 890 for receiving the electrical power required for the operation of the illumination modules.

The secondary modular imaging unit 805 may also comprise protruding elements such as protruding elements 822 and 824 configured to attach the secondary modular imaging unit 805 to the primary modular imaging unit (not shown). In some cases, the protruding elements 822 and 824 may be inserted into corresponding slots or grooves at the primary modular imaging unit. In such cases, closing the distal tip by attaching the secondary modular imaging unit 805 and inserting the protruding elements 822 and 824 into the corresponding slots or grooves may form a unified, firmed and steady distal tip, such configured to detachably fit within detachable fitting element/s of the primary modular imaging unit 305. For example, detachable fitting element/s 371 of primary modular imaging unit 305 as described with reference to FIG. 3B. The secondary modular imaging unit 805 further comprise an edge 820 of its U shape. Edge 820 comprises a secondary side upper closing margin 871 and a secondary side lower closing margin 872. The secondary side upper closing margin 871 and the secondary side lower closing margin 872 can be fastened to corresponding closing margins of the primary modular imaging unit. In some cases, fastening the secondary side upper closing margin 871 and the secondary side lower closing margin 872 may seal the distal tip and prevent leaking of liquids, body fluids and gases into the distal tip. In some cases, the two protruding elements 822 and 824 may not be inserted into any slots or grooves. Thus, the two protruding elements 822 and 824 may be squeezed into the longitudinal opening of the primary modular imaging unit.

In some possible embodiments of the disclosed subject matter, secondary modular imaging unit 805 may comprise more than two protruding elements, in some other possible embodiments the protruding elements may be slots, rail shaped slots, grooves, or other mechanisms which can ensure a strong fastening of the secondary modular imaging unit 805 within the primary modular imaging unit. In some other cases, the protruding elements may be placed on the primary modular imaging unit. In such cases, the secondary modular imaging unit 805 may comprise the slots, rail shaped slots, grooves, or other mechanisms which can ensure a strong fastening of the secondary modular imaging unit 805 within the primary modular imaging unit. In some embodiments, the number and the location of the protruding elements may vary from 1 to 10 or more along the margins of edge 820. In other embodiments, the secondary modular imaging unit 805 may fit into longitudinal opening or void of the primary modular imaging unit without any protruding elements.

FIG. 8C shows an exploded view of a secondary modular imaging unit, which can function as a bracket combined into a primary modular imaging unit, according to FIGS. 8A-8B. FIG. 8C shows a secondary modular imaging unit 805 designed to fit into a longitudinal opening or void of a primary modular imaging unit to create a cylinder or a cylinder like geometric figure adapted to a shape of a distal tip for example distal tip 205 of a multi camera medical imaging device 207 as described with reference to FIG. 2. In such cases, the secondary modular imaging unit 805 can function as a lid to a primary modular imaging unit (not shown) and thereby to form a unified structure of a distal tip (not shown).

The secondary modular imaging unit 805 may comprise a U-shape having a second side niche 860 located on a lateral side parallel to longitudinal axis 897 of secondary modular imaging unit 805 and designed to house a second side camera optical window 850. The second side camera optical window 850 is adapted to be inserted within a second side camera aperture 852 and to cover a second side lens assembly 831. The second side camera aperture 852 configured to hold, support and secure second side lens assembly 831 within the lateral surface of secondary modular imaging unit 805. The second side camera optical window 850 is adapted to provide the opening required for the field of view of the second side lens assembly 831 and of a second side sensor 880. In some cases, the second side camera optical window 850 may comprise a transparent layer, such as glass or plastic, to isolate the second side lens assembly 831 from liquids, gases and patient's debris and tissue. In some cases, the second side camera optical window 850 may comprise semi-transparent layer allowing a part of the light spectrum to pass out from the second side camera optical window 850. In some cases, the second side camera optical window 850 may comprise a light filter to block a portion of the light spectrum from pass to the second side lens assembly 831 and the second side sensor 880

The secondary modular imaging unit 805 also comprise a second side illuminator optical window 845 which can be inserted into a second side illuminator opening 843, and a second side illuminator optical window 855 which can be inserted into a second side illuminator opening 853. The second side camera aperture 852 is typically located between second side illuminator opening 843 and second side illuminator opening 853 within the secondary modular imaging unit 805 lateral surface. Typically, second side camera aperture 852 and second side illuminator openings 843 and 853 are aligned along the longitudinal axis 897 of the secondary modular imaging unit 805 within second side niche 860.

In some embodiments, the second side illuminator optical windows 845 and 855 may comprise a transparent layer, such as glass or plastic, to isolate the second side illuminator/s from liquids, gases and patient's debris and tissue. In some other embodiments, second side illuminator optical windows 845 and 855 may comprise an optical window or more than one optical window. The second side illuminator optical windows 845 and 855 enable emission of light emitted by second side illumination modules 841 and 851, respectively

Such, a second side camera comprises the second side lens assembly 831 and the second side sensor 880 may provide a field of view between 80 to 130 degrees and a working distance in a range of 1-30 millimeters, in a range of 5-150 millimeters, wherein the second side illumination modules 841 and 851 adapted to illuminate the field of view of the second side camera.

In some embodiments, the second side illuminator optical windows 845 and 855, and the second side camera optical window 850 may be connected to the second side illuminator openings 843 and 853 and to second side camera aperture 852, respectively, by adhesive material, screws, soldering, clamping devices, and the like. In some embodiments, second side illuminator optical windows 845 and 855, and the second side camera optical window 850 may be attached to second side illuminator openings 843 and 853 and to second side camera aperture 852, respectively, in detachable manner techniques, this enables the replacement of second side illumination modules 841 and 851 or any illuminator within these modules and some of the lenses of the second side lens assembly 831.

The U-shape of secondary modular imaging unit 805 designed to allow the required freedom for inserting, arranging, and in some cases, replacing a second side optical gear. The second side optical gear can be mounted on a second side foldable circuit board 890 comprising a second side longitudinal circuit board 893 positioned in parallel to the secondary modular imaging unit longitudinal axis 897 and extends outward from a rear end section 825 of secondary modular imaging unit 805 into the primary modular imaging unit (not shown). The second side foldable circuit board 890 further comprise a second side rigid circuit board 863 that connect to the second side longitudinal circuit board 893 along longitudinal axis 897 and parallel to the second side niche 860. The second side rigid circuit board 863 is designed to carry the second side sensor 880 and the second side lens assembly 831. The structure of second side foldable circuit board 890 may allow locating the second side sensor 880 and the second side lens assembly 831 (e.g., the second side camera) within the secondary modular imaging unit 805, in such way that second side lens assembly 831 is aligned with the second side camera aperture 852.

The secondary modular imaging unit 805 further comprise a second side illuminator electronic circuit board 876 adapted to hold second side illumination modules 841 and 851 in place. The second side illuminator electronic circuit board 876 has a “U” opening 877 shaped to accommodate and secure the second side lens assembly 831 there within. Thus, the second side illuminator electronic circuit board 876 can be positioned within the secondary modular imaging unit 805 such that the “U” opening 877 is adapted to aligned with the second side camera aperture 852. The second side illuminator optical windows 845 and 855 may configured to hold, support and secure the second side illuminator electronic circuit board 876 within the lateral surface of secondary modular imaging unit 805.

In some embodiments, the second side illuminator electronic circuit board 876 can hold a set of two second side illumination modules, represented by second side illumination modules 841 and 851. In some embodiments, second side illuminator electronic circuit board 876 can hold a set of 1, 2, 3, 4, 5 or more second side illumination modules. In other possible embodiments, the number and location of second side illuminators modules may vary for example, less than two second side illumination modules or more, wherein each second side illumination modules may hold 1, 2, 3, 4 or more LED. In some cases, said LED's may emit light at the same light spectrum. In some cases, said LED's may emit light at different light spectrum.

The second side lens assembly 831 may comprise a set of lenses employed to capture light and transmit the captured light to the second side sensor 880. The second side lens assembly 831 may define a field of view, which can be the field of view captured by the second side optical gear of the secondary modular imaging unit 805. Optionally, the set of lenses of the second side lens assembly 831 and second side sensor 880 is configured to have a field of view of at least 80 degrees and up to essentially 135 degrees, and a working distance of at least 1 millimeters and up to essentially 150 millimeters,

The secondary modular imaging unit 805 may also comprise protruding elements represented by protruding elements 822 and 824 located at the margins of edge 820 of the secondary modular imaging unit 805. The protruding elements 822 and 824 may configured to fit and fasten the secondary modular imaging unit 805 to the primary modular imaging unit (not shown), results in a sealed closing between the two modular imaging units that essentially prevent entry of debris fluids and or gases to inner parts of the isolated two modular imaging units. In some cases, protruding elements 822 and 824 may be inserted into corresponding slots or grooves located within longitudinal opening or void of the primary modular imaging unit. In other embodiments, protruding elements 822 and 824 are slots or grooves adapted to protruding elements within the primary modular imaging unit. In some embodiments, protruding elements 822 and 824 of secondary modular imaging unit 805 configure to detachably fit within detachable fitting element/s of the primary modular imaging unit.

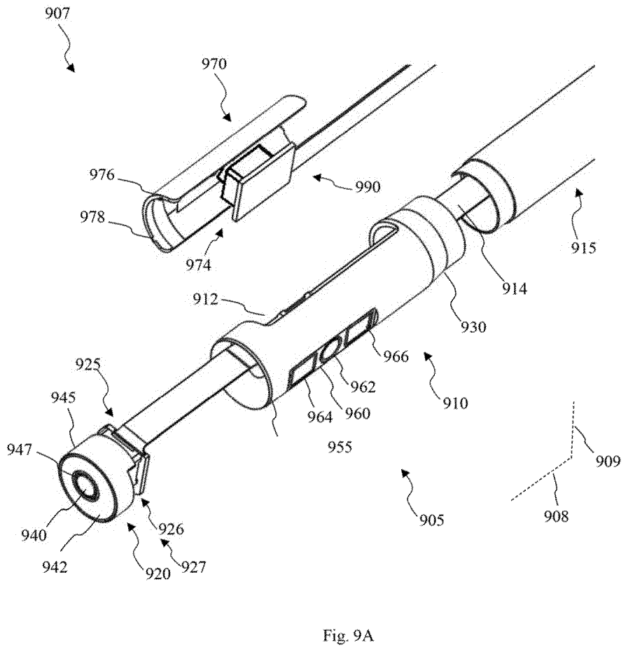

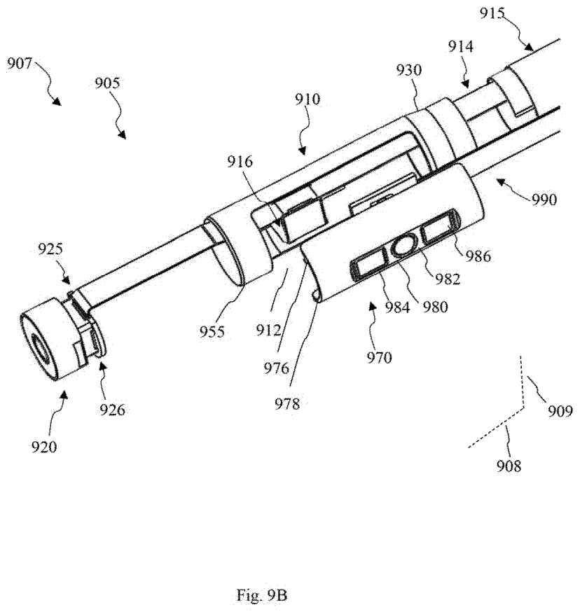

FIG. 9A shows a first side perspective view of a distal tip of a multi camera medical imaging device comprising three imaging units, shown in an exploded view, according to exemplary embodiments of the disclosed subject matter. FIG. 9A shows a multi camera medical imaging device 907 comprising a distal tip 905 and a rigid shaft 915. The distal tip 905 comprises a primary modular imaging unit 910, a secondary modular imaging unit 970 and a front modular imaging unit 920. The three modular imaging units 910, 970 and 920 are adapted to interlocked with each other and fit over inner parts of the distal tip 905, and to provide protection to internal components within the inner parts. The internal components within the inner parts of the distal tip 905 may comprise lens assemblies, sensors, foldable circuit boards, illuminator electronic circuit boards, illuminator modules, and additional circuits and electrical components to convey and control the electrical signals and the electrical power required for the operation of optical gears. The primary modular imaging unit 910, the secondary modular imaging unit 970 and the front modular imaging unit 920 are configured to abut to cover the distal tip 905 of the multi camera medical imaging device 907.

The primary modular imaging unit 910 has a longitudinal opening or void 912, the longitudinal opening 912 aligns with the secondary modular imaging unit 970 U-shape design, such that, secondary modular imaging unit 970 may function as a bracket along the longitudinal opening 912. The distal tip 905 further comprise the front modular imaging unit 920 adapted to abut within a distal front end 955 of the primary modular imaging unit 910.

The secondary modular imaging unit 970 comprises a second side foldable circuit board 990 for holding and supporting a second side optical gear 974 located within the secondary modular imaging unit 970 parallel to longitudinal axis 908. In some cases, a user may utilize the secondary modular imaging unit 970 as a bracket by closing protruding elements 976 and 978 of the secondary modular imaging unit 970 within the longitudinal opening 912. Closing the longitudinal opening 912 by secondary modular imaging unit 970 may introduce a cylinder geometric figure or a cylinder like geometric figure to the distal tip 905.

The primary modular imaging unit 910 comprise a niche 960 designed to house a first side camera optical window 962 and first side illuminator optical windows 964 and 966. Niche 960 is located on the lateral area of the primary modular imaging unit 910 parallel to the longitudinal opening 912 and to longitudinal axis 908. The primary modular imaging unit 910 further comprise a first side foldable circuit board 914 for holding and supporting a first side optical gear (not shown). The first side foldable circuit board 914 is placed parallel to the longitudinal axis 908 and adapted to support a first side camera (not shown) and a first side illuminator electronic circuit board (not shown) which designed to host first side illumination modules (not shown). The first side illumination modules are adapted to provide the required light source for the functioning of a first side sensor (not shown) and to enable emission of light for illuminating the area captured by the first side camera (not shown).

According to some embodiments, a second side camera of secondary modular imaging unit 970 and the first side camera may be placed such that their field of views are substantially opposing. However, different configurations between the side cameras are possible within the general scope of the current invention. In some embodiments, the second side optical gear 974 and the first side optical gear may be directed to opposing sides. However, different configurations between the side optical gears are possible within the general scope of the current invention. The first side camera may be positioned at the opposite side of the second side camera such that the two cameras may be pointing at directions essentially opposing to one another. The center of the first side camera may be approximately 3 to 18 millimeters from the distal front end 955 of the primary modular imaging unit 910. Second side camera may be located such that the center of second side camera may be located approximately 0.0 to 10.0 millimeters from the center of first side camera.

The front modular imaging unit 920 comprises a front frame 945 designed to house a front camera optical window 940 and a front illuminator optical window 942. The front camera optical window 640 is adapted to cover a front camera 926 and to be inserted within a front frame camera aperture 947. The front modular imaging unit 920 further comprise a front foldable circuit board 925 for holding and supporting a front optical gear 927 provides a front field of view of the multi camera medical imaging device 907. The front foldable circuit board 925 is placed parallelly to longitudinal axis 908 and enables the location of the front optical gear 927 along axis 909 essentially in a vertical position or almost a vertical position to longitudinal rods 908. The front modular imaging unit 920 further comprise a front illuminator electronic circuit board (not shown) designed to host the front illumination modules (not shown) which provide the required light source to a front camera 926.

Engaging the three modular imaging units 910, 920 and 970 may introduce a cylinder geometric figure or a cylinder like geometric figure, e.g., distal tip 905. The distal tip 905 has a sealed distal end equipped with front modular imaging unit 920 and a sealed interlocking with the rigid shaft 915. Distal tip 905 is adapted to provide the multi camera medical imaging device 907 a field of view along longitudinal axis 908 and axis 909. A first side field of view along longitudinal axis 908 is captured by the first side camera placed within the primary modular imaging unit 910, a second side field of view along longitudinal axis 908, yet oppose the first side field of view, is captured by the second side camera placed within the secondary modular imaging unit 970, and a front field of view along axis 909, such orthogonal or almost orthogonal to longitudinal axis 908, is captured by the front camera placed within the front modular imaging unit 920. Optionally, the three cameras are similar or identical, however different camera designs may be used, for example, field of view, depth of work, focal length and the like may be different. In some embodiments, the first side field of view and the front field of view may comprise a first overlap between the field of views, and the second side field of view and the front field of view may comprise a second overlap between the field of views. The two overlaps' field of views may be the same or different. The first side camera may be positioned at the opposite side of the second side camera such that the two cameras may be pointing at directions essentially opposing to one another. The center of the first side camera may be approximately 5 to 15 millimeters from the center of the front camera optical window 940. Second side camera located such that the center of second side camera may be located approximately 0.0 to 10.0 millimeters from the center of first side camera. U.S. Provisional Patent Application No. 62/546,581, which relates to the Application of the present specification, titled “Multi camera medical surgery illuminating device with a changing diameter” and filed on Aug. 17, 2017, is one example of differences between the location the first side camera and the second side camera relatively to the front camera and is herein incorporated by reference in its entirety.

In some cases, connecting the three modular imaging units 910, 920 and 970 may be supported by adhesives. In some other cases, soldering, mechanical device, magnetic connectors, and the like may be utilized to support the engagement and adapted to autoclave requirements.

The primary modular imaging unit 910 may comprise an inclined surface 930 allowing to connect the distal tip 905 to the rigid shaft 915 provided in a narrower diameter than the diameter of the distal tip 905. In some embodiments, the first side foldable circuit board 914, the second side foldable circuit board 990 and the front foldable circuit board 925 may be positioned in parallel to longitudinal axis 908 and extends outward from the inclined surface 930 of primary modular imaging unit 910 into the rigid shaft 915. FIG. 9B shows a second side perspective view of a distal tip, according FIG. 9A. FIG. 9B shows a multi camera medical imaging device 907 comprising a distal tip 905 and a rigid shaft 915. The distal tip 905 comprises a primary modular imaging unit 910, a secondary modular imaging unit 970 and a front modular imaging unit 920. The three modular imaging units 910, 970 and 920 are adapted to interlocked with each other and to fit over inner parts of the distal tip 905, and to provide protection to internal components in the inner part from outer fluids and gases.

The secondary modular imaging unit 970 comprises a U-shape having a second side niche 980 designed to house a second side camera optical window 982 and second side illuminator optical windows 984 and 986. Second side niche 980 is located on the lateral area of the U-shaped secondary modular imaging unit 970 parallel to a longitudinal opening or void 912 of the primary modular imaging unit 910, and also parallel to the longitudinal axis 908. The secondary modular imaging unit 970 further comprise a second side foldable circuit board 990 for holding and supporting a second side optical gear (not shown) located parallel to the longitudinal axis 908. The secondary modular imaging unit 970 is also adapted to support a second side illuminator electronic circuit board (not shown) designed to host the second side illumination modules (not shown) which provide the required light source to the second side optical gear.

In some cases, the longitudinal opening 912 of the primary modular imaging unit 910 aligns with the secondary modular imaging unit 970 U-shape design, such that secondary modular imaging unit 970 may function as a bracket by closing protruding elements 976 and 978 of the secondary modular imaging unit 970 within the longitudinal opening 912. Closing the longitudinal opening 912 by secondary modular imaging unit 970 may introduce a cylinder geometric figure or a cylinder like geometric figure to the distal tip 905.

The primary modular imaging unit 910 also comprise a first side foldable circuit board 914 for holding and supporting a first side optical gear 916 located within the primary modular imaging unit 910 parallel to the longitudinal axis 908. The first side foldable circuit board 914 is also adapted to support a first side illuminator electronic circuit board (not shown) designed to host a first side illumination module (not shown) which provide the required light source to the first side optical gear 916.

In some cases, attaching the front modular imaging unit 920 and secondary modular imaging unit 970 to the primary modular imaging unit 910 may be supported by soldering and or adhesives. In some other cases, mechanical device, magnetic connectors, and the like may be utilized to support the attachment of the front modular imaging unit 920 and secondary modular imaging unit 970 to the primary modular imaging unit 910.

In some embodiments, the first side foldable circuit board 914, the second side foldable circuit board 990 and the front foldable circuit board 925 may be positioned in parallel to the longitudinal axis 908 and extends outwards from an inclined surface 930 of primary modular imaging unit 910 into the rigid shaft 915.

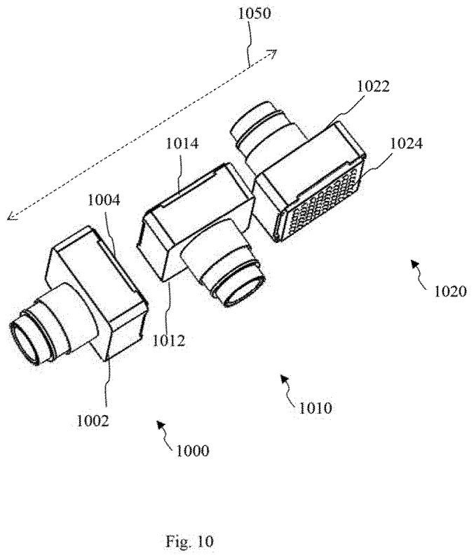

FIG. 10 shows a front and two side cameras located within the distal tip, where the front, primary and secondary modular imaging units shown in FIGS. 1 and 9A are removed. FIG. 10 shows a front camera 1000 (shown as 633 and 926 in FIGS. 6 and 9, respectively), a first side camera 1010 (shown as part of first side optical gear 333 and 916 in FIGS. 3B-4 and 9B, respectively) and a second side camera 1020 (shown as part of second side optical gear 833 and 974 in. FIGS. 8B and 9A, respectively) position as when placed within front, primary and secondary modular imaging units, respectively in accordance with an embodiment of the present specification. As shown, front camera 1000 comprises a lens assembly 1002 and a sensor 1004, first side camera 1010 comprises a lens assembly 1012 and a sensor 1014, and second side camera 1020 comprises a lens assembly 1022 and a sensor 1024.

In an embodiment, the three cameras, each comprises a lens assembly associated with a sensor as shown in FIGS. 9A-9B, are assembled within a distal tip (not shown) of a multi camera medical imaging device (not shown). Front camera 1000 positioned parallel to a longitudinal axis 1050 Raced in a direction of the distal tip and adapted to provide a front field of view for the multi camera imaging device. Once the front camera 1000 is assembled within the distal tip, it faces forward and in an outward direction when viewed with respect to the center of the distal tip. First side camera 1010 associated with a first side field of view and second side camera 1020 associated with a second side field of view. The two side cameras 1010 and 1020 are positioned one after the other along longitudinal axis 1050 such that their field of views are facing in opposite directions or in essentially opposite directions.

According to some embodiments, an overlap between the front field of view and each of the sides field of view may occur, such overlap may be adapted to provide a surround view to a user of the multi camera medical imaging device. The two overlaps' field of views may be the same or different. As illustrated along longitudinal axis 1050, first side camera 1010 is located closer to front camera 1000 while second side camera 1020 is placed further away from front camera 1000. In another embodiment, second side camera 102 may be placed closer to front camera 1000.

In some embodiments, each camera, for example—the front camera 1000, the first side camera 1010 and the second side camera 1020, may provide a field of view between 80 to 130 degrees and a working distance in a range of 1 to 30 millimeters, in a range of 3 to 150 millimeters. In some embodiment, front, and each of the side cameras have the same sensors and lens assemblies, yet in other embodiments, the cameras may be different, such the front camera and each of the side cameras may be the same or different in any one or any combinations of their components or other element related to them (such as optical elements).

In some embodiments, the front camera 1000 may situated at the center of the distal tip, either on a flat surface perpendicular to longitudinal axis 1050 or on a tilted front surface and such create an angle smaller than 90 degrees from the longitudinal axis 1050. The center of the first side camera 1010 may be approximately 3 to 35 millimeters from a distal front end of the front modular imaging unit. Second side camera 1020 may be located such that the center of second side camera 1020 may be located approximately 0.1 to 10.0 millimeters from the center of first side camera 1010.

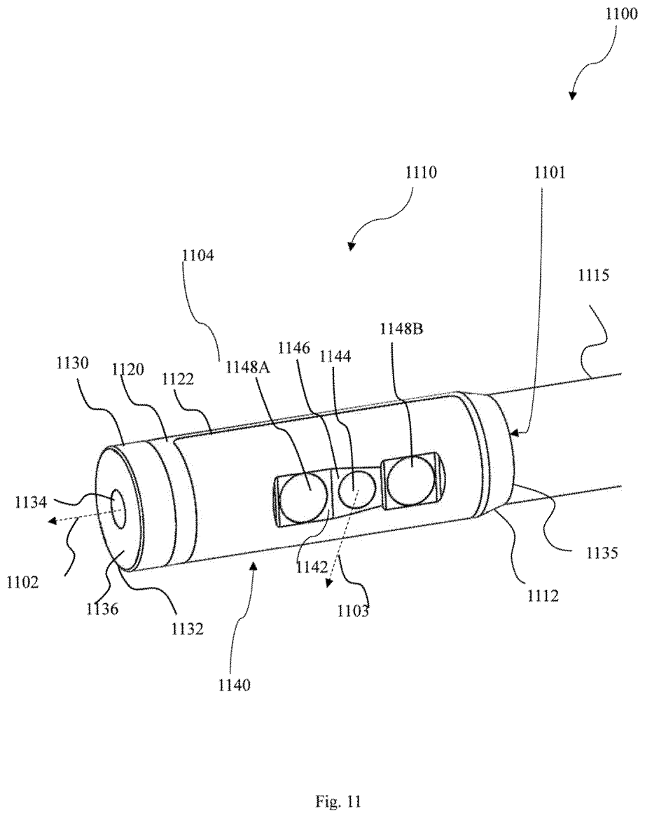

FIG. 11 schematically depicts a medical imaging device 1100 comprising at least one tilted camera and a changing diameter according to a second exemplary embodiment of the current specification.

In contrast to the first embodiment depicted in FIGS. 1 through 10, in the embodiment depicted in FIGS. 11 through 16C, at least one of the three cameras located within a distal tip 1110 of medical imaging device 1100 is tilted towards a direction of view of a front camera.

FIG. 11 shows multi camera medical imaging device 1100 comprising a distal tip 1110 adapted to be connected to a rigid shaft 1115 at a rear/distal end section 1101. The distal tip 1110 may comprise an inclined surface 1112 (as inclined surface 230 of FIG. 2) allowing to connect the distal tip 1110 to the rigid shaft 1115. The rigid shaft 1115 can be provided in a diameter which is narrower than the diameter of the distal tip 1110. The multi camera medical imaging device 1100 also comprises a seamline 1135 outlining the connection in between the rigid shaft 1115 and the inclined surface 1112 of the distal tip 1110 (as seamline 235 of FIG. 2). In some cases, the rigid shaft 1115 and the distal tip 1110 may be connected at the seamline 1135 by an adhesive material, which seals the connection at the seamline 1135. In some cases, the rigid shaft 1115 and the distal tip 1110 may be connected at the seamline 1135 by soldering. In some other cases, the rigid shaft 1115 and the distal tip 1110 may be connected by an adhesive material which seals the connection at the seamline 1135. In possible embodiments of the disclosed subject matter, the rigid shaft 1115 and the distal tip 1110 may be connected by a screwing mechanism which fastens the rigid shaft 1115 and the distal tip 1110 together. In some embodiments of the disclosed subject matter, the inclined surface may be configured to be replaced by other inclined surfaces with different inclination levels for allowing the distal tip 1110 to be directly connected with rigid shafts having other diameters.

The distal tip 1110 may function as a multi-camera section member designed to house at least two cameras. In some cases, of the at least two cameras at least one camera may be positioned at the front end of the distal tip 1110 abutting a front lens optical window 1134. Other cameras may be located at the lateral surfaces of the distal tip 1110. The distal tip 1110 may comprise a primary modular imaging unit 1120 designed to be connected to the rigid shaft 1115. The primary modular imaging unit 1120 has a longitudinal opening 1122. The longitudinal opening 1122 may be adapted to be aligned with a secondary modular imaging unit 1140, such that the secondary modular imaging unit 1140 may function as a bracket enclosing the longitudinal opening 1122 of primary modular imaging unit 1120 along the length thereof. The multi camera medical imaging device 1100 further comprises a front modular imaging unit 1130 abutting a front end 1132 of the primary modular imaging unit 1120.

The three imaging units, primary modular imaging unit 1120, front modular imaging unit 1130 and secondary modular imaging unit 1140, form the distal tip 1110. In some cases, the secondary modular imaging unit 1140 and the front modular imaging unit 1130 may be attached to the primary modular imaging unit 1120 in a sealed fashion, which prevents leaking of liquids, gases and/or debris and or body fluids into the distal tip 1110. In some cases, the secondary modular imaging unit 1140 and the front modular imaging unit 1130 may be attached to the primary modular imaging unit 1120 by an adhesive material, soldering, screwing mechanism and the like. In some embodiments of the disclosed subject matter, the secondary modular imaging unit 1140 may be detachable from the primary modular imaging unit 1120. In some cases, a second optical gear located within the distal tip 1110 may be mounted onto the secondary modular imaging unit 1140, such that when detaching and pulling out the primary modular imaging unit 1120, the second optical gear may also be pulled out jointly with the modular secondary imaging unit 1140.

The secondary modular imaging unit 1140 may comprise a niche 1142 designed to house a second side camera optical window 1144 and provide the opening required for the field of view of a second side camera (not shown). In some cases, the second side camera optical window 1144 may comprise a transparent layer, such as glass or plastic, to isolate the second side camera from liquids, gases and patient's debris and tissue. In some other cases, second side camera may be covered by an optical window or more than one optical window. The niche 1142 also enables emission of light from second side illuminator optical windows 1148A and 1148B for illuminating the area captured by the second side camera. In some cases, the light may be emitted by dedicated section illuminators such as light-emitting diodes, also known as LED. The dedicated section illuminators may be housed within the second side illuminator optical windows 1148A and 1148B.

In some embodiments of the disclosed subject matter, the second side niche 1142 may comprise a second side tilted camera platform 1146. The second side tilted camera platform 1146 may be structured with some inclination outwards from the second side niche 1142. The inclined structure of the second side tilted camera platform 1146 allows the second side camera optical window 1144 to be tilted towards the direction of view 1102 of the front camera (not shown) and abutting the front lens optical window 1134. In some cases, the second side camera optical window 1144 may be located at the second side tilted camera platform 1146. The tilt of the second side camera optical window 1144 can allow a direction of view 1103 of a second camera (not shown) to be tilted relative to the direction of view 1102 of the front camera (not shown) located at the primary modular imaging unit 1120, as elaborated below. In such cases, a direction of view such as directions of views 1102 and 1103 are defined as a straight and imaginary line extending from the focal point of the lens assembly, through the center of the lens assembly (not shown). In some cases, the direction of view 1102 of the front camera located within the primary modular imaging unit 1120 may be non-perpendicularly to the direction of view 1103 of a second side camera (not shown) located within the secondary modular imaging unit 1140. In such cases, the tilt of the second side tilted camera platform 1146 tilts the second camera, and thereby causes the direction of view 1103 to be tilted towards the direction of view 1102. Namely, an angle created between an imaginary line which continues the direction of view 1102 and an imaginary line which continues the direction of view 1103 is less than 90 degrees. PCT Patent Application No. PCT/IL2018/050826, which relates to the Application of the present specification, titled “A Two-piece rigid medical surgery illuminating device” and filed on Jul. 25, 2018, is one example of tilted side cameras and is herein incorporated by reference in FIGS. 7-8 and related disclosure.

In some embodiments of the disclosed subject matter, the front modular imaging unit 1130 may comprise a front surface 1136 designed to hold, secure and seal the front lens optical window 1134 within. The front lens optical window 1134 may be adapted to provide with the opening required for capturing a front field of view of the front camera. In some cases, the front side camera optical window 1134 may comprise a transparent layer, such as glass or plastic., to isolate the front side optical gear from liquids, gases and patient's debris and tissue situated outside of the distal tip 1110. In some cases, the front optical gear may be covered by more than one camera optical window.

In some embodiments of the disclosed subject matter, the primary modular imaging unit 1120 may comprise a first side niche (not shown) located along the length of the lateral side of the primary modular imaging unit 1120. In some cases, the first side niche is adapted to hold a first side camera optical window (not shown) which covers, protects and provides the opening required for capturing the first side field of view by a first side camera (not shown). The first side camera optical window may comprise a transparent layer, such as glass or plastic, to isolate the first side optical gear from liquids, gases and patient's debris and tissue. In some cases, first side optical gear may be covered by more than one camera optical window. In some cases, the first side niche is designed to hold at least one first side illuminator optical window (not shown) for enabling emission of light from at least one first side illuminator (not shown). In some cases, the at least one first side illuminator may by a dedicated section illuminator such as light-emitting diodes, also known as LED. In other embodiments, the first side niche may be covered by a transparent layer, such as glass or plastic or an optical window or more than one optical window for isolating the first side niche from liquids, gases and patient's debris and tissue.

In possible embodiments of the subject matter, the front surface 1136 may also designed as a front illuminator optical window for enabling emission of light from at least one front illuminator. For example, the front modular imaging unit 1130 may comprise a front illuminator optical window shaped as a ring around the front lens optical window 1134 and may span on about the front end 1132. In some cases, the at least one front illuminator may be dedicated section illuminator such as LED. In some embodiments of the disclosed subject matter, most of the front surface 1136 of the front modular imaging unit 1130 may be covered by a transparent layer, such as glass or plastic or an optical window or more than one optical window for isolating the front modular imaging unit 1130 from liquids, gases and patient's debris and tissue.

In some embodiments of the disclosed subject matter, the distal tip 1110 can be structured to allow one or more overlapping views formed by the fields of views captured by the lens assemblies of the distal tip 1110. The term “overlapping views” as disclosed herein refers to a converged area wherein at least one object captured by one lens assembly, is also captured simultaneously by at least one other lens assembly. Hence, the fields of views of the optical gears at least partly overlaps. in some embodiments of the disclosed subject matter, the at least one of the side cameras and the front camera of the multi camera medical imaging device are configured to have overlapping views with a sufficiently narrow convergence angle. The overlapping views which may be achieved by at: least two lens assembly. In such cases, the lens assembly may be adapted to provide the overlapping views at a working distance.

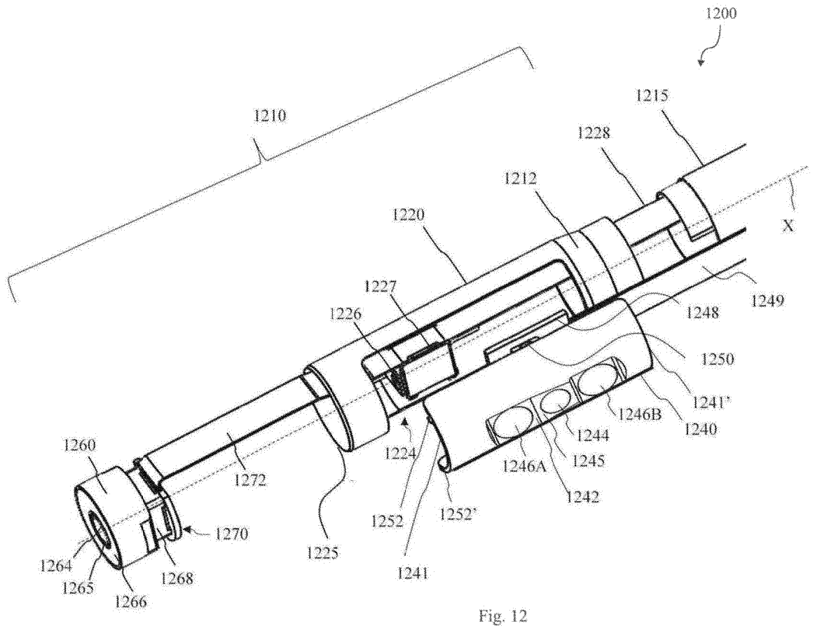

FIG. 12 shows a side perspective view of a distal tip of a multi camera medical imaging device comprising three modular imaging units, according to FIG. 11. FIG. 12 shows a multi camera medical it device 1200 comprising a distal tip 1210 and a rigid shaft 1215. The distal tip 1210 comprises a primary modular imaging unit 1220, a secondary modular imaging unit 1240 and a front modular imaging unit 1260. The time: imaging units, primary modular imaging unit 1220, front modular imaging unit 1260 and secondary modular imaging unit 1240, form the distal tip 1210 as described in association with FIGS. 9A-9B with the modification described in FIG. 11.

The primary modular imaging unit 1220 comprise a niche (not shown) designed to house a first side optical gear 1226, wherein first side niche is designed to hold a first side camera optical window, and first side illuminator optical windows. In some cases, the first side niche may comprise a first side tilted camera platform, in such cases, the first side tilted camera platform is structured with inclination relatively to longitudinal axis X of distal tip 1210, as elaborated below. The first side camera optical window is resided at the first side tilted camera platform.

The secondary modular imaging unit 1240 is formed in a U-shape having a second side niche or a second side shallow trough 1242, extending in parallel to the longitudinal axis X. The second side niche 1242 is designed to hold a second side camera optical window 1244, and second side illuminator optical windows 1246A and 1246B. In some cases, the second side niche 1242 may comprise a second side tilted camera platform 1245. In some cases, the second side tilted camera plattbrm 1245 is structured with inclination outwards relatively to second side illuminator optical windows 1246A placed also in the second side niche 1242, as elaborated below, in some cases, the second side camera optical window 1244 is resided at the second side tilted camera platform 1245. In some cases, the second side camera optical window 1244 is configured to cover a second side optical gear 1250 which is situated within the secondary modular imaging unit 1240. The second side camera optical window 1244 is further configured to protect the second side optical gear 1250 from liquids, gases, debris and tissue that may be located on the exterior side of the distal tip.

In some cases, the second side optical gear 1250 comprises a second side foldable circuit comprising a second side rigid circuit board section 1248 and a second side longitudinal circuit board section 1249. The second side rigid circuit board section 1248 may be designed to support and hold the second side optical gear 1250 of the secondary modular imaging unit 1240. The second side longitudinal circuit board section 1249 can be located in parallel to the longitudinal axis X and is configured to extend from the second side rigid circuit board section 1248 to the rigid shaft 1215.

In some cases, the longitudinal opening 1224 of the primary modular imaging unit 1220 aligns with the secondary modular imaging unit 1240 U-shape design, such that the secondary modular imaging unit 1240 may function as a bracket designed to enclose the longitudinal opening 1224. In some cases, the secondary modular imaging unit 1240 may enclose the primary modular imaging unit 1220 by inserting a protruding element 1252 located an a front/proximal side 241 of the secondary modular imaging unit 1240 within the longitudinal opening 1224. In some cases, the secondary modular imaging unit 1240 may enclose the primary modular imaging unit 1220 by inserting a protruding element 1252′ located on a rear/distal side 1241′ of the secondary modular imaging unit 1240 within the longitudinal opening 1224. In other cases, the secondary modular imaging unit 1240 may enclose the primary modular imaging unit 1220 by inserting the protruding element 1252 located on front/proximal side 1241 and the protruding element 1252′ located on rear/distal side 1241′ within the longitudinal opening 1224. In some cases, the secondary modular imaging unit 1240 may be used as a bracket by closing protruding element 1252 of the secondary modular imaging unit 1240 within the longitudinal opening 1224. Closing the longitudinal opening 1224 by secondary modular imaging unit 1240 may introduce a cylinder geometric figure or a cylinder like geometric figure to the distal tip 1210.

In some embodiments of the disclosed subject matter, the front modular imaging unit 1260 is designed as a short hollow tube and may hold a front lens optical window 1264 and at least one front illuminator optical window 1266. The front modular imaging unit 1260 further comprises a front optical gear 1268.

The front modular imaging unit 1260 further comprises a front circuit board comprising a front rigid circuit board section 1270 and a front longitudinal circuit board section 1272. The front rigid circuit board section 1270 is designed to hold the front optical gear 1268 required for capturing the front field of view. The front longitudinal circuit board section 1272 can be located in parallel to the longitudinal axis X and configured to extend from the front modular imaging unit 1260 to the rigid shaft 1215. In some cases, the front modular imaging unit 1260 aligns with the front end 1225 of the primary modular imaging unit 1220 such that, the front modular imaging unit 1260 may function as a bracket which encloses the front end 1225.

The primary, secondary and front modular imaging units 1220, 1240 and 1260 can be adapted to jointly form a cylinder-shaped distal tip 1210. In some cases, the primary modular imaging unit 1220, secondary modular imaging unit 1240, and the front modular imaging unit 1260 may be jointly connected by adhesives. Such adhesives may isolate the optical gears within the distal tip 1210 and prevent ingress of liquids, gases and patient's debris and tissue. In some other cases, soldering, mechanical device, magnetic connectors, and the like may be utilized to jointly connect the modular imaging units for meeting autoclave requirements. In some cases, the primary modular imaging unit 1220 may comprise an inclined surface 1214 allowing the primary modular imaging unit 1220 to connect the distal tip 1210 to the rigid shaft 1215 at the rear/distal side of the distal tip 1210, in cases the rigid shaft 1215 is provided in a narrower diameter than the diameter of the distal tip 1210.

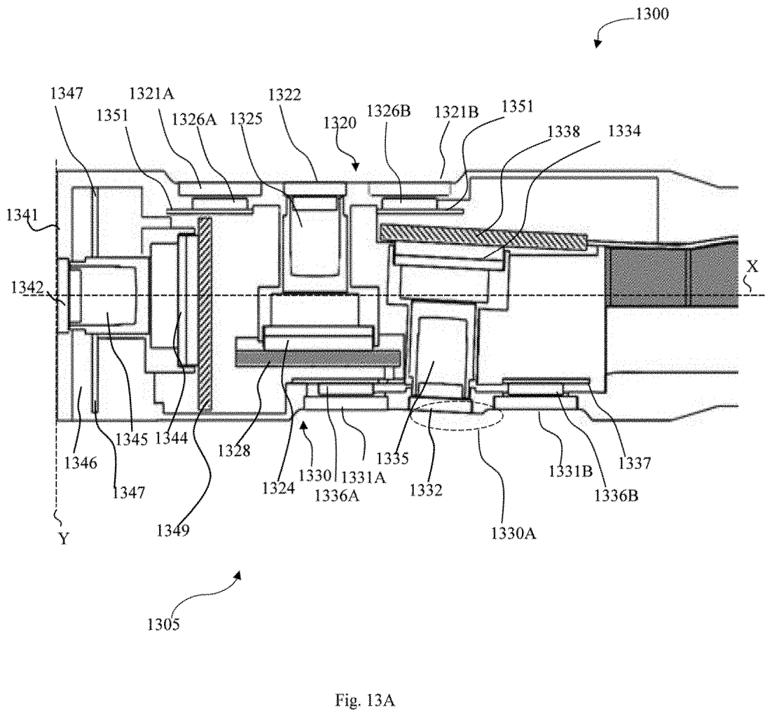

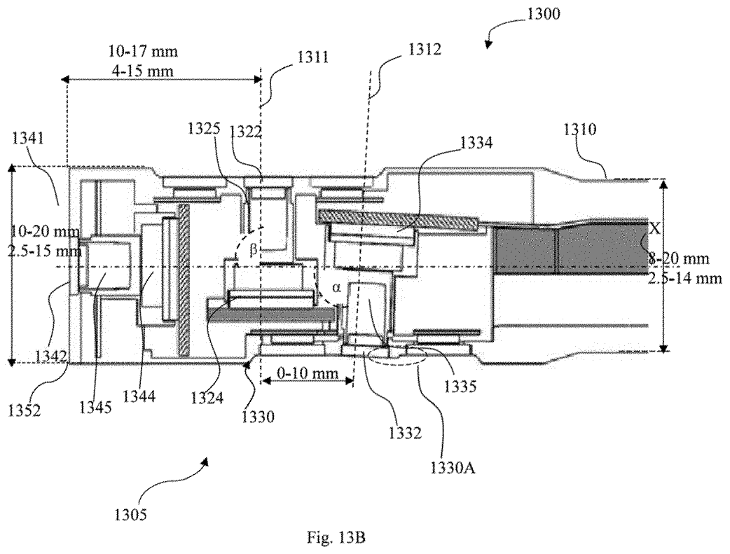

FIG. 13A demonstrates a cross-sectional view of a distal tip of a multi camera medical imaging device with a tilted secondary lens assembly and sensor, according to FIG. 11. FIG. 13A shows a distal tip 1305 of a multi camera medical imaging device 1300 designed to be connected to a rigid shaft 1310. The distal tip 1305 comprises a first optical gear comprising alter alia a first side lens assembly 1325 pointing at the direction of a first side camera optical window 1322. The first side lens assembly 1325 can be located at the primary modular imaging unit, for example primary modular imaging unit 1220 of a distal tip 1210 as described with reference to FIGS. 11-12. The distal tip 1305 also comprises a first side niche or a first side niche or shallow trough 1320 adapted to house, hold and seal the first side camera optical window 1322 therein. The first side niche 1320 positioned on a lateral side of the primary modular imaging unit extends in parallel to the distal tip 1305 longitudinal axis X. The first side niche 1320 design to house a first side illuminator optical window 1321A and a first side illuminator optical window 1321B. In some embodiments, the first side illuminator optical windows 1321A and 1321B may comprise a transparent layer, such as glass or plastic, to isolate first side illuminator/s from liquids, gases and patient's debris and tissue. In some other embodiments, first side illuminator optical windows 1321A and 1321B may comprise an optical window or more than one optical window. The first side illuminator optical windows 1321A and 1321B enable emission of light emitted by first side illumination modules 1326A and 1326B, respectively.

The distal tip 1305 further comprises a first side sensor 1324 configured to convert the light captured by the first side lens assembly 1325 to electrical signals in a form of current. In some cases, the first side sensor 1324 may be connected to a first side rigid circuit board 1328, of a first side foldable circuit board (not shown), for conveying the electrical signals to an external electronic device (not shown) designed to receive such electrical signals. The distal tip 1305 may also comprise a first side illuminator electronic circuit board 1351. The first side illuminator electronic circuit board 1351 designs to host and support the first side illumination modules 1326A and 1326B. In some embodiments of the disclosed subject matter, the electronic signals conveyed from the first side sensor 1324 to the first side foldable circuit board via the first side rigid circuit board 1328 may represent the light captured by the first side lens assembly 1325 and converted to current by the first side sensor 1324. The first side rigid circuit board 1328 may be able to convey communications between the first side sensor 1324 and the first side lens assembly 1325 to, and/or from the first side longitudinal circuit board. The conveyed communications may be such as electrical signals representing the light captured b the first side lens assembly 1325 and required for the operation of the first side sensor 1324, first side lens assembly 1325, first side illumination modules 1326A and 1326B, and first side illuminator electronic circuit board 1351. For example, first side optical gear, digitized data as a result of the first side optical gear operation, and the like. In some cases, electrical power may also be conveyed via the first side foldable circuit board to the first side optical gear. In some embodiments of the disclosed subject matter, the number and location of first side illumination modules 1326A and 1326B may vary for example, less than two first side illumination modules or more, wherein the first side illumination module may hold 1, 2, 3, 4 or more LED. In some cases, said LED's may emit light at the same light spectrum. In cases, said LED's may emit light at different light spectrum.

In some cases, the first side illuminator optical window 1321A may be located on one side of the first side camera optical window 1322 and the first side illuminator optical window 1321B may be located on another side of the first side camera optical window 1322. In one embodiment, the center of the first side camera optical window 1322 is placed in the center of first side niche 1320 equally apart from the first side illuminator optical windows 1321A and 1321B. In another embodiment, the center of the first side camera optical window 1322 is placed within the first side niche 1320 such that the distance between the first side illuminator optical window 1321A and the center of the first side camera optical window 1322 may be unequal to the distance between the first side illuminator optical window 1321B and the center of the first side camera optical window 1322. In such cases, the first side illuminator optical windows 1321A and 1321B and the first side camera optical window are situated on the same plane of the first side niche 1320, and in parallel to the longitudinal axis X.

The first side lens assembly 1325 may comprise a set of lenses employed to capture light and transmit the captured light to the first side sensor 1324. The first side lens assembly 1325 may capture and determinate a first side field of view, which can be the field of view captured by the primary modular imaging unit. Optionally, the set of lenses of the first side lens assembly 1325, and first side sensor 1324 are configured to have a field of view of at least 80 degrees and up to essentially 135 degrees, and a working distance of at least 1 millimeter and up to essentially 150 millimeters.

The distal tip 1305 further comprises a second side optical gear comprising alter alia a second side lens assembly 1335 pointing at the direction of a second side camera optical window 1332. The second side lens assembly 1335 can be located at the secondary modular imaging unit, for example secondary modular imaging unit 1240 of a distal tip 1210 as described with reference to FIG. 12. The distal tip 1305 also comprises a second side niche or a second side shallow trough 1330 adapted to house, hold and seal the second side camera optical window 1332 therein. The second side niche 1330 positioned on a lateral side of the secondary modular imaging unit parallel to the distal tip 1305 longitudinal axis X. The second side niche 1330 design to house a second side illuminator optical window 1331A and a second side illuminator optical window 1331B. In some embodiments, the second side illuminator optical windows 1331A and 1331B may comprise a transparent layer, such as glass or plastic, to isolate first side illuminator/s from liquids, gases and patient's debris and tissue. In some other embodiments, second side illuminator optical windows 1331A and 1331B may comprise an optical window or more than one optical window. The second side illuminator optical windows 1331A and 1331B enable emission of light emitted by second side illumination modules 1336A and 1336B, respectively.

In some cases, the second side illuminator optical window 1331A may be located on one side of the second side camera optical window 1332 and the second side illuminator optical window 1331B may be located on another side of the second side camera optical window 1332. In one embodiment, the center of the second side camera optical window 1332 is placed in the center of second side niche 1330 equally apart from the second side illuminator optical windows 1331A and 1331B. In another embodiment, the center of the second side camera optical window 1332 is placed within the second side niche 1330 such that the distance between the second side illuminator optical window 1331A and the center of the second side camera optical window 1332 may be unequal to the distance between the second side illuminator optical window 1331B and the center of the second side camera optical window 1332. In such cases, the second side illuminator optical windows 1331A and 1331B are situated on the same plane of the second side niche 1330, and in parallel to the longitudinal axis X, whereas the second side camera optical window 1332 may be situated tilted to the second side niche 1330 plane.

The distal tip 1305 further comprise a second side sensor 1334 configured to convert the light captured by the second side lens assembly 1335 to electrical signals in a form of current. In some cases, the second side sensor 1334 may be connected to a second side rigid circuit board 1338, of a second side foldable circuit board, for conveying the electrical signals to an external electronic device (not shown) designed to receive such electrical signals. The distal up 1305 may also comprise a second side illuminator electronic circuit board 1337. The second side illuminator electronic circuit board 1337 designs to host and support the second side illumination modules 1336A and 1336B. In some embodiments, the second side sensor 1334 may configure to communicate the electronic signals to the second side foldable circuit board via the second side rigid circuit board 1338. The second side rigid circuit board 1338 may be able to conduct the communications between the second side sensor 1334 and the second side lens assembly 1335 to, and/or from the second side longitudinal circuit board. The conducted communications may be such as electrical signals required for the operation of the second side sensor 1334, second side lens assembly 1335, second side illumination modules 1336A and 1336B, and second side illuminator electronic circuit board 1337, e.g., second side optical gear, digitized data as a result of the second side optical gear operation, and the like. In some cases, electrical power may also be conveyed via the second side foldable circuit board to the second side optical gear.

In some embodiments of the disclosed subject matter, the number and location of second side illuminator modules 1336A and 1336B may vary for example, less than two second side illumination modules or more, wherein the second side illumination module may hold 1, 2, 3, 4 or more LED. In some cases, said LED's may emit light at the same light spectrum. In cases, said LED's may emit light at different light spectrum.

The second side lens assembly 1335 may comprise a set of lenses employed to capture light and transmit the captured light to the second side sensor 1334. The second side lens assembly 1335 may capture and determine a second side field of view, which can be the field of view captured by the secondary modular imaging unit. Optionally, the set of lenses of the second side lens assembly 1335, and second side sensor 1334 are configured to have a field of view of at least 80 degrees and up to essentially 135 degrees, and a working distance of at least 1 millimeter and up to essentially 150 millimeters.

The distal tip 1305 further comprises a front optical gear comprising alter alia front lens assembly 1345 pointing at the direction of a front lens optical window 1342. The front lens assembly 1345 can be located at a front modular imaging unit, for example front modular imaging unit 1260 of a distal tip 1210 as described with reference to FIG. 12. The front optical gear may also comprise a front sensor 1344 coupled with the front lens assembly 1345, configured to receive the light captured by the front lens assembly 1345 and to convert that light to electric signals in a form of current. The front optical gear further comprises a front illumination module 1346 located on a front illuminator electronic circuit board 1347. The front illuminator electronic circuit board 1347 is situated within the distal tip 1305 such that the front illumination module 1346 is situated abutting to the front illuminator optical windows 1341. The front modular imaging unit further comprises a front circuit board comprising a front rigid circuit board 1349. The front rigid circuit board 1349 may be configured to support and secure the front optical gear components such as the front lens assembly 1345 and the front sensor 1344. In some cases, the front rigid circuit board 1349 is connected via a front longitudinal circuit board section extending from the front rigid circuit board 1349 into the rigid shaft 1310.

In some embodiments of the disclosed subject matter, the number and location of front illumination module 1341 may vary for example, more than one front illumination module, wherein the front illumination module may hold 1, 2 3, 4 or more LED. In some cases, said LED's may emit light at the same light spectrum. In cases, said LED's may emit light at different light spectrum. The front lens assembly 1345 may comprise a set of lenses employed to capture light and transmit the captured light to the front sensor 1344. The front lens assembly 1345 may capture and determinate a front field of view, which can be the field of view captured by the front modular imaging unit. Optionally, the set of lenses of the front lens assembly 1345, and front sensor 1344 are configured to have a field of view of at least 80 degrees and up to essentially 135 degrees, and a working distance of at least 1 millimeter and up to essentially 150 millimeters.

In some embodiments of the disclosed subject matter, the multi camera medical imaging device 1300 may be structured to allow differences in the distance between the center of the first side lens assembly 1325 and the center of the front lens assembly 1345, and in the distance between the center of the second side lens assembly 1335 and the center of the front lens assembly 1345. Thus, the distance between center of the first side lens assembly 1325 and an axis Y may be shorter than the distance between center of the second side lens assembly 1335 and axis Y, wherein longitudinal axis Y is perpendicular to longitudinal axis X and parallel to front lens window 1342. Yet, in some embodiments, the distance between center of the first side lens assembly 1325 and axis Y may be longer than the distance between center of the second side lens assembly 1335 and axis Y.

In possible embodiments of the disclosed subject matter, the distal tip 1305 may be structured with the second side niche 1330 comprising a second side tilted camera platform 1330A. The second side tilted camera platform 1330A may be provided in a dedicated tilted structure which positions at least one of the second side camera's components in a tilt/elevate position. Such, as the second side tilted camera platform 1330A positions in a dedicated tilted structure the second side camera optical window 1332 of the second side camera may be positioned in a tilt position, also, as the second side tilted camera platform 1330A positions in a dedicated tilted structure the second side camera optical window 1332 and the second side lens assembly 1335 of the second side camera may be positioned in a tilt position also, as the second side tilted camera platform 1330A positions in a dedicated tilted structure the second side camera optical window 1332, the second side lens assembly 1335 and the second side sensor 1334 of the second side camera may be positioned in a tilt position, moreover, as the second side tilted camera platform 1330A positions in a dedicated tilted structure the second side camera optical window 1332, the second side lens assembly 1335 and the second side sensor 1334 of the second side camera, and the second side rigid circuit board 1338 may be positioned in a tilt position and thereby allows an object to be seen in more than one lens assembly simultaneously. Thus, the second side tilted camera platform 1330A can situate at least one of the second side camera's components in a position wherein an imagery straight line following a direction of view of the second side lens assembly 1335 and an imagery straight line following a direction of view of the front lens assembly 1345 may be non-perpendicularly, as elaborated further below. For example, overlapping views may be formed by the view captured by the second side lens assembly 1335 and the view captured by the front lens assembly 1345. The second side lens assembly 1335 and the front lens assembly 1345 may be adapted to provide the overlapping views at the working distance. In some cases, the tilt of the second side lens assembly 1335 may also enable the overlapping views between the front camera comprising a front lens assembly and the first side camera comprising a first side lens assembly to align with the overlapping views between the front camera and the second side camera, wherein the center of the first side lens assembly of the first side camera is placed in a first distance from the center of the front lens assembly of the front camera, and the center of the second side lens assembly of the second side camera is placed in a second distance from the center of the front lens assembly of the front camera and wherein the first distance is shorter than the second distance.

FIG. 13B shows a cross-sectional view of a distal tip of a multi camera medical imaging device with a tilted secondary lens assembly, according: to FIG. 13A. FIG. 13B shows the distal tip 1305 connected to the rigid shaft 1310. The distal tip 1305 comprises a distal front end 1352 designed to hold a front illuminator optical window 1341 for allowing emission of light from at least one front illuminator module. In some embodiments of the disclosed subject matter, the diameter of the distal tip 1305 ranges between 10 to 20 millimeters. In possible embodiments of the disclosed subject matter, the diameter of the distal tip 1305 range of 2.5 to 15 millimeters.

In possible cases, the diameter of the rigid shaft 1310 may be provided in different sizes according to some specific patterns of the subject matter. In some cases, the diameter of the rigid shaft 1310 connected to the distal tip 1305 may be with a narrow diameter compare to the distal tip 1305 and to range of 8 millimeters to 20 millimeters. In some other cases, the diameter of the rigid shaft 1310 can be in the range of 2.5 millimeters to 14 millimeters. The term diameter refers to the cross-sectional diameter of the distal tip 1305 and the cross-sectional diameter of the rigid shaft 1310.

The distal tip 1305 which may be shaped as a hollow tube can comprise front lens assembly 1345 situated such that the center of the front lens assembly 1345 is overlapped with the center of the tube-shaped distal tip 1305. The front lens assembly 1345 may be situated abutting to a front lens optical window 1342. The front lens assembly 1345 may be coupled with a front sensor 1344 configured to receive the light captured by the front lens assembly 1345 and convert that light to electric signals in a form of current.

The distal tip 1305 also comprises first side optical gear comprises a first side lens assembly 1325 which can be situated abutting to a first side camera optical window 1322. The first side optical gear may also comprise a first side sensor 1324 coupled with the first side lens assembly 1325, configured to receive the light captured by the first side lens assembly 1325 and to convert that light to electric signals in a form of current. In some embodiments of the disclosed subject matter, the length measured from the first side camera optical window 1322 to the first side sensor 1324 is longer than the radius of the distal tip 1305. In such embodiments, the length of the first side camera is longer than the radius of the distal tip 1305.

In possible embodiments of the disclosed subject matter, the first side lens assembly 1325 may be positioned such that a first direction of view 1311 is pointed vertically to the distal tip 1305 longitudinal axis X. The first direction of view 1311 is defined as a straight and imaginary line extending from the center of the first side lens assembly 1325 through the focal point of the first side lens assembly 1325. Thus, a first side angle β representing the angle between the first direction of view 1311 and the longitudinal axis X may be essentially 90 degrees. In some cases, the distance between the center of first side lens assembly 1325 and the distal front end 1352 may be approximately 10 to 17 millimeters. In some other cases, the distance between the center of the first side lens assembly 1325 and the distal front end 1352 may be approximately 4 to 15 millimeters.

The distal tip 1305 further comprises a first side optical gear comprises a second side lens assembly 1335 which can be situated abutting to a second side camera optical window 1332. The second side optical gear may also comprise a second side sensor 1334 coupled with the second side lens assembly 1335, configured to receive the light captured by the second side lens assembly 1335 and to convert that light to electric signals in a form of current. In some embodiments of the disclosed subject matter, the length measured from the second side camera optical window 1332 to the second side sensor 1334 is longer than the radius of the distal tip 1305. In such embodiments, the length of the second side camera is longer than the radius of the distal tip 1305.

The distal tip 1305 may be structured with a diameter which dominates the locations of the first side lens assembly 1325 and the second side lens assembly 1335. In some cases, the distal tip 1305 diameter may dominate the location of a first side camera (not shown) comprising the first side lens assembly 1325, and the location of the second side camera (not shown) comprising second side lens assembly 1335. The lengths measured from the side lens optical windows through the side lens assemblies to the side sensors may cause the side lens assemblies to be located one after the other and not back to back. Locating the side cameras one after the other will cause the directions of views of the cameras to be not aligned.

The distal tip 1305 further comprises a second side niche 1330 comprising a second side tilted camera platform 1330A. The second side tilted camera platform 1330A may be provided in a dedicated tilted structure which situates the second side lens assembly 1335 in a tilt position, and thereby allows an object to be captured by more than one lens assembly simultaneously. The second side lens assembly 1335 may be positioned with a second direction of view 1312 tilted relative to the distal tip 1305 longitudinal axis X due to the structure of the second side tilted camera platform 1330A. Thus, a second side angle α representing the angle between the second direction of view 1312 and the longitudinal axis X can be in a range of 80 to 90 degrees. The second direction of view 1312 is defined as a straight and imaginary line extending from the center of the second side lens assembly 1335 through the focal point of the first side lens assembly 1325. In some cases, the distance between the second direction of view 1312 and the first direction of view 1311 is between 0 to 10 millimeters.

In some cases, the tilt of the second side lens assembly 1335 may cause to the field of view captured by the second side lens assembly 1335 and the field of view captured by the front lens assembly 1345 to contain some overlapping views. For example, the direction of view 1312 may be tilted such that, an object captured by the front lens assembly 1345 may simultaneously be captured by the second side lens assembly 1335 along the working distance of each lens assembly. In such cases, the second side camera comprising the second side lens assembly 1335 and the front camera comprising the front lens assembly 1345 may have overlapping views. In some cases, the tilt of the second side lens assembly 1335 may also enable the overlapping views between the front camera comprising the front lens assembly 1345 and the first side camera comprising the first side lens assembly 1325 to align with the overlapping views between the front lens assembly 1345 and the second side lens assembly 1335, wherein the first side lens assembly 1325 is placed in a first distance from the front lens assembly 1345 and the second side lens assembly 1335 is placed in a second distance from the front lens assembly 1345 and wherein the first distance is shorter than the second distance.

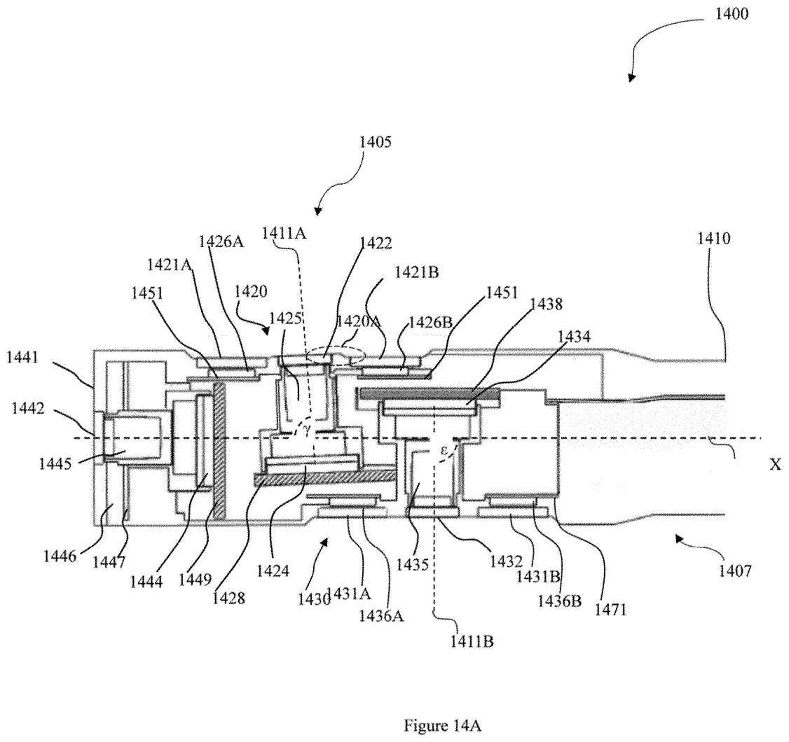

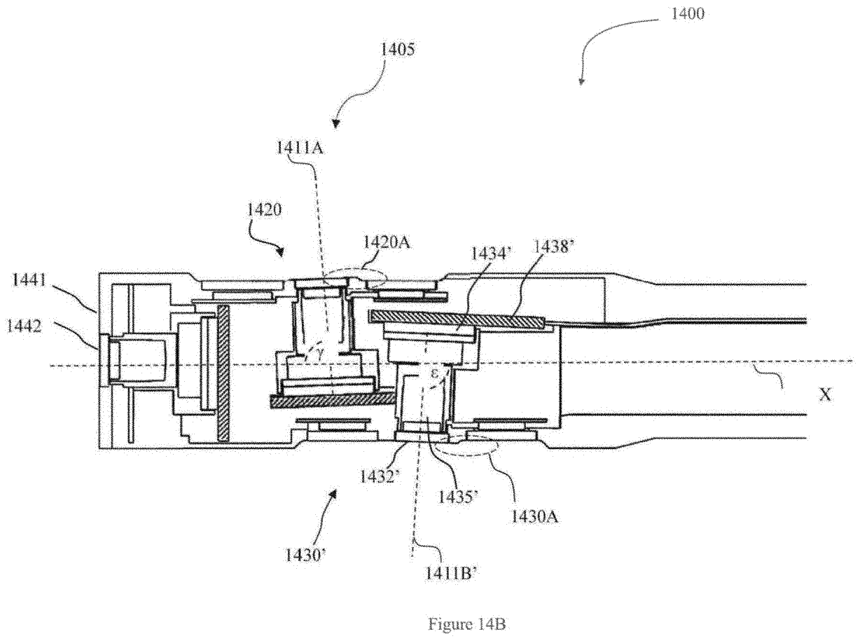

FIG. 14A shows cross-sectional view of a multi camera medical imaging device with a tilted first lens assembly, according exemplary embodiments of the disclosed subject matter. FIG. 14A shows a distal tip 1405 of a multi camera medical imaging device 1400 designed to be connected to a rigid shaft 1410. The distal tip 1405 comprises a first side niche or first side shallow trough 1420 designed to house a first side lens assembly 1425 pointing at the direction of a first side camera optical window 1422. The first side niche 1420 positioned on a lateral side of a primary modular imaging unit along distal tip 1405 longitudinal axis X, for example primary modular imaging unit 1220 of a distal tip 1210 as described with reference to FIG. 12. The first side niche 1420 may comprise a first side tilted camera platform 1420A. The first side camera tilted platform 1420A may be provided in a dedicated structure which situates the first side camera optical window 1422 in a tilt/elevate, and thereby allow one object to be captured by more than one lens assembly, simultaneously. The first side tilted camera platform 1420A can be tilted at a first side angle γ, wherein the first side angle γ is measured between the distal tip 1405 longitudinal axis X and a first direction of view 1411A. The first direction of view 1411A may be defined as a straight and imaginary line extending from the center of the first side lens assembly 1425 through the focal point of the first side lens assembly 1425. In some cases, the first side angle γ, represents the angle between the first direction of view 1411A and the longitudinal axis X and can be in a range of 80 to 90 degrees.

The distal tip 1405 may also comprise a first side sensor 1424 configured to convert the light captured by the first side lens assembly 1425 to electrical signals in a form of current. In some cases, the first side sensor 1424 may be connected to a first side rigid circuit board 1428 for conveying the electrical signals to an external electronic device (not shown) designed to receive electrical signals. The first side rigid circuit board 1428 may configured to communicate with a first side optical gear associated with the first side lens assembly 1425 and first side sensor 1424.

The first side niche 1420 is further designed to house a first side illuminator optical window 1421A and a first side illuminator optical window 1421B. In some embodiments, the first side illuminator optical windows 1421A and 1421B may comprise a transparent layer, such as glass or plastic, to isolate first side illuminator/s from liquids, gases and patient's debris and tissue. In some other embodiments, first side illuminator optical windows 1421A and 1421B may comprise an optical window or more than one optical window. The first side illuminator optical windows 1421A and 1421B enable emission of light emitted by first side illumination modules 1426A and 1426B, respectively.

The distal tip 1405 further comprise a first side illuminator electronic circuit board 1451 configured to support the illumination modules 1426A and 1426B. In some cases, the first side illuminator optical window 1421A may be located at one side of the first side camera optical window 1422 and the first side illuminator optical window 1421B may be located on the other side of the first side camera optical window 1422. In one embodiment, the center of the first side camera optical window 1422 is placed in the center of first side niche 1420 equally apart from the first side illuminator optical windows 1421A and 1421B. In another embodiment, the center of the first side camera optical window 1422 is placed within the first side niche 1420 such that the distance between the first side illuminator optical window 1421A and the center of the first side camera optical window 1422 may be unequal to the distance between the first side illuminator optical window 1421B and the center of the first side camera optical window 1422. In such cases, the first side illuminator optical windows 1421A and 1421B are situated on the same plane of the first side niche 1420, and in parallel to the longitudinal axis X, whereas the first side camera optical window 1422 may be situated tilted to the first side niche 1420 plane.

The first side tilted camera platform 1420A may be provided in a dedicated tilted/elevated structure which positions at least one of the first side camera's components in a tilt position. Such, as the first side tilted camera platform 1420A positions in a dedicated tilted structure the first side camera optical window 1422 may be positioned in a tilt position, also, as the first side tilted camera platform 1420A positions in a dedicated tilted stnicture the first side camera optical window 1422 and the first side lens assembly 1425 of the first side camera may be positioned in a tilt position also, as the first side tilted camera platform 1420A positions in a dedicated tilted structure the first side camera optical window 1422, the first side lens assembly 1425 and the first side sensor 1424 of the first side camera may be positioned in a tilt position, moreover, as the first side tilted camera platform 1420A positions in a dedicated tilted structure the first side camera optical window 1422, the first side lens assembly 1425 and the first side sensor 1424 of the first side camera, and the first side rigid circuit board 1428 may be positioned in a tilt position. Thus, the first side tilted camera platform 1420A can situate at least one of the first side camera's components in a position wherein an imagery straight line following the direction of view of the first side lens assembly 1425 and an imagery straight line following direction of view of a front lens assembly 1445 may be non-perpendicularly, as elaborated further below. For example, overlapping views may be formed by the view captured by the first side lens assembly 1425 and the view captured by the front lens assembly 1445. The first side lens assembly 1425 and the front lens assembly 1445 may be adapted to provide the overlapping views at the working distance. In some cases, the tilt of the first side lens assembly 1425 may also enable the overlapping views between the view captured by the front lens assembly 1445 and the view captured by first side lens assembly 1425 to align with the overlapping views between the view captured by the front lens assembly 1445 and the view captured by a second side lens assembly 1435, wherein the first side lens assembly 1425 is placed in a first distance from the front lens assembly 1445 and the second side lens assembly 1435 is placed in a second distance from the front lens assembly 1445 and wherein the first distance is shorter than the second distance.

The distal tip 1405 further comprises the first side rigid circuit board 1428 configured to communicate with some optical gear components required for the operation of the first side camera. For example, in case the first side camera's components required for the operation of the first side lens assembly 1425 and the first side sensor 1424. In such an exemplary case, the first side rigid circuit board 1428 may convey the power required for the operation of the first side sensor 1424. The first side sensor 1424 can be configured to convert the light captured by the first side lens assembly 1425 to electrical signals in a form of current. In such cases, the first side sensor 1424 may be connected to the first side rigid circuit board 1428 for conveying the electrical signals to an external electronic unit (not shown) designed to receive such electrical signals. The first side rigid circuit board 1428 also may be able to conduct the communications between the first side sensor 1424 and the first side lens assembly 1425 to, and/or from a first side longitudinal circuit board. The conducted communications may be such as, electrical signals required for the operation of the first side sensor 1424, first side lens assembly 1425, first side illumination modules 1426A and 1426B, and first side illuminator electronic circuit board 1451, e.g., first side optical gear, digitized data as a result of the first side optical gear operation, and the like. In some cases, electrical power may also be conveyed via the first side foldable circuit board to the first side optical gear. In some embodiments of the disclosed subject matter, the number and location of first side illumination modules 1426A and 1426B may vary for example, less than two first side illumination modules or more, wherein the first side illumination module may hold 1, 2. 3, 4 or more LED. In some cases, said LED's may emit light at the same light spectrum. In cases, said LED's may emit light at different light spectrum.

The distal tip 1405 further comprises a second side lens assembly 1435 with a second direction of view 1411B pointing at the direction of a second side camera optical window 1432. The second side lens assembly 1435 can be located at a secondary modular imaging unit, for example secondary modular imaging unit 1240 of a distal tip 1210 as described with reference to FIG. 12. The distal tip 1405 also comprises a second side niche 1430 adapted to hold, secure and seal the second side camera optical window 1432 within. The second direction of view 1411B is defined as a straight and imaginary line extending from the center of the second side lens assembly 1435 through the focal point of the second side lens assembly 1435. The second side lens assembly 1435 can point forward such that a second side angle ε measured between the distal zip 1405 longitudinal axis X and the second direction of view 1411B can be essentially 90 degrees. The second side niche 1430 positioned on a lateral side of the secondary modular imaging unit extends in parallel to the distal tip 1405 longitudinal axis X. The second side niche 1430 can also be adapted to house secure and seal a second side illuminator optical window 1431A and a second side illuminator optical window 1431B there within. The second side illuminator optical windows 1431A and 1431B are designed to emit the light required for the operation of the second side camera.

In some cases, the second side illuminator optical window 1431A may be located on one side of the second side camera optical window 1432 and the second side illuminator optical window 1431B may be located on another side of the second side camera optical window 1432. In one embodiment, the center of the second side camera optical window 1432 is placed in the center of second side niche 1430 equally apart from the second side illuminator optical windows 1431A and 1431B. In another embodiment, the center of the second side camera optical window 1432 is placed within the second side niche 1430 such that the distance between the second side illuminator optical window 1431A and the center of the second side camera optical window 1432 may be unequal to the distance between the second side illuminator optical window 1431B and the center of the second side camera optical window 1432. In such cases, the second side illuminator optical windows 1431A and 1431B and the second side camera optical window 1432 are situated on the same plane of the second side niche 1430, and in parallel to the distal tip 1405 longitudinal axis X.

The distal tip 1405 further comprises a second side rigid circuit board 1438 configured to communicate with some optical gear components required for the operation of the second side lens assembly 1435, which in some cases may reside within a second side camera. For example, in case the optical gear components required for the operation of the second side camera comprises the second side lens assembly 1435 and a second side sensor 1434, in such an exemplary case, the second side rigid circuit board 1438 may convey the power required for the operation of the second side sensor 1434. The second side sensor 1434 can be configured to convert the light captured by the second side lens assembly 1435 to electrical signals in a form of current. In such cases, the second side sensor 1434 may be connected to the second side rigid circuit board 1438 for conveying the electrical signals to an external electronic unit (not shown) designed to receive such electrical signals. The second side rigid circuit board 1438 also may be able to convey the communications between the second side sensor 1434 and the second side lens assembly 1435 to, and/or from a second side longitudinal circuit board. The conveyed communications may be such as, digital signals, and/or electrical signals required for the operation of the second side sensor 1434, second side lens assembly 1435, first side illumination modules 1436A and 1436B, and second side illuminator electronic circuit board 1471, e.g., first side optical gear, digitized data as a result of the second side optical gear operation, and the like. In some cases, electrical power may also be conveyed via the second side foldable circuit board to the second side optical gear. In some embodiments of the disclosed subject matter, the number and location of second side illuminators modules 1436A and 1436B may vary for example, less than two first side illumination modules or more, wherein the second side illumination module may hold 1, 2, 3, 4 or more LED. In some cases, said LED's may emit light at the same light spectrum. In cases, said LED's may emit light at different light spectrum.

The distal tip 1405 further comprises a front lens assembly 1445 pointing at the direction of a front lens optical window 1442. The front lens assembly 1445 can be located at a front modular imaging unit, for example front modular imaging unit 1260 of a distal tip 1210 as described with reference to FIG. 12. The front lens assembly 1445 may be coupled with a front sensor 1444 configured to receive the light captured by the front lens assembly 1445 and convert that light to electric signals in a form of current. The distal tip 1405 also comprises at least one front illumination module 1446 located on a front illuminator electronic circuit board 1447. The front illuminator electronic circuit board 1447 is situated within the distal tip 1405 such that the front illumination module 1446 is situated abutting to at least one front illuminator optical windows 1441. In some embodiments of the disclosed subject matter, the number and location of front illumination module 1446 may vary for example, more than one front illumination module, wherein the front illumination module may hold 1, 2, 3, 4 or more LED. In some cases, said LED's may emit light at the same light spectrum. In cases, said. LED's may emit light at different light spectrum.

A front circuit board comprising a front rigid circuit board 1449 and a front longitudinal circuit board section (not shown). The front rigid circuit board 1449 can be configured to support and secure optical gear components such as the front lens assembly 1445 and the front sensor 1444. In such an exemplary case, the front rigid circuit board 1449 may convey the power required for the operation of the front sensor 1444. The front sensor 1444 can be configured to convert the light captured by the front lens assembly 1445 to electrical signals in a form of current. In such cases, the front sensor 1444 may be connected to the front rigid circuit board 1449 for conveying the electrical signals to an external electronic unit (not shown) designed to receive such electrical signals. The front rigid circuit board 1449 also may be able to conduct the communications between the front sensor 1444 and the front lens assembly 1445 to, and/or from the front longitudinal circuit board. The conducted communications may be such as, digital video signals, electrical signals required for the operation of the front sensor 1444, front lens assembly 1445, from illumination module 1446 and front illuminator electronic circuit board 1447, e.g., front optical gear, digitized data as a result of the front optical gear operation, and the like. In some cases, electrical power may also be conveyed via the front foldable circuit board to the front optical gear. In some embodiments, the distal tip 1405 comprises the first side niche 1420 having the first side tilted camera platform 1420A and a second side niche 1430′ having a second side tilted camera platform 1430A, as shown in FIG. 14B.

FIG. 14B shows cross-sectional view of a multi camera medical imaging device with a tilted first lens assembly 1425 and a tilted second side lens assembly 1435′, according to exemplary embodiments of the disclosed subject matter. Thus, the first side camera optical window 1422 and a second side camera optical window 1432′ may be situated in a tilt/elevate angle relatively to distal tip 1405 longitudinal axis X, and thereby allow one object to be captured by more than one lens assembly, simultaneously.

The first side tilted camera platform 1420A can be tilted at a first side angle γ, wherein the first side angle γ is measured between the distal tip longitudinal axis X and a first side camera direction of view 1411A. The first direction of view 1411A may be defined as a straight and imaginary line extending from the center of the first side lens assembly 1425 through the focal point of the first side lens assembly 1425. In some cases, the first side angle γ, represents the angle between the first direction of view 1425 and the longitudinal axis X and can be in a range of 80 to 90 degrees. The second side tilted camera platform 1430A can be tilted at a second side angle ε, wherein the second side angle ε is measured between the distal tip longitudinal axis X and a second side camera direction of view 1411B′. The second direction of view 1411B′ may be defined as a straight and imaginary line extending from the center of the second side lens assembly 1435′ through the focal point of the second side lens assembly 1435′. In some cases, the second side angle ε, represents the angle between the second direction of view 1411B′ and the longitudinal axis X and can be in a range of 80 to 90 degrees.

In some cases, the tilt of the first side lens assembly 1425 may also enable the overlapping views between the front camera and the first side camera to align with the overlapping views between the front camera and the tilted second side lens assembly 1435′ wherein such alignment between the two overlapping views may provide a panoramic view along the working distance of the three cameras. Such, the first side tilted camera platform 1420A tilted/elevated in first side angle γ and the second side tilted camera platform 1430A tilted/elevated in second side angle ε and wherein the first side angle γ is smaller than the second side angle ε. In some possible embodiments of the disclosed subject matter, the second side tilted camera platform 1430A may be tilted/elevated relatively to the longitudinal axis X in an angle of about 0.5 to 10 degrees, of about 0.5 to 4.5 degrees, of about 1.5 to 3.5 degrees, of about 2.0 to 3.0 degrees. In some possible embodiments of the disclosed subject matter, the first side tilted camera platform 1420A may be tilted/elevated relatively to the longitudinal axis X in an angle of about 0.5 to 10 degrees, of about 0.5 to 4.5 degrees, of about 1.5 to 3.5 degrees, of about 2.0 to 3.0 degrees.

In other embodiments of the disclosed subject matter the distal tip 1405 may be designed to house the front lens assembly 1445 in a tilted position (not shown). In such embodiments, the front lens optical window 1442 may be tilted such that, the direction of view of the front lens assembly 1445 may not be parallel to the distal tip 1405 longitudinal axis X. The front direction of view of the front lens assembly 1445 may be defined as a straight and imaginary line extending from the center of the front lens assembly 1445 through the focal point of the front lens assembly 1445. In some cases, wherein the front lens assembly 1445 is tilted, the direction of view 1411A and the direction of view 1411B are essentially parallel to the longitudinal axis X. In some cases, wherein the front lens assembly 1445 is tilted, the direction of view 1411A of first side lens assembly 1425 is essentially parallel to the longitudinal axis X wherein the direction of view 1411B of second side lens assembly 1435 and the longitudinal axis X can be in a range of 80 to 90 degrees. In some cases, wherein the front lens assembly 1445 is tilted, the direction of view 1411A of first side lens assembly 1425 can be in a range of 80 to 90 degrees to the longitudinal axis X, wherein the direction of view 1411B of second side lens assembly 1435 and the longitudinal axis X is essentially parallel to the longitudinal axis X.

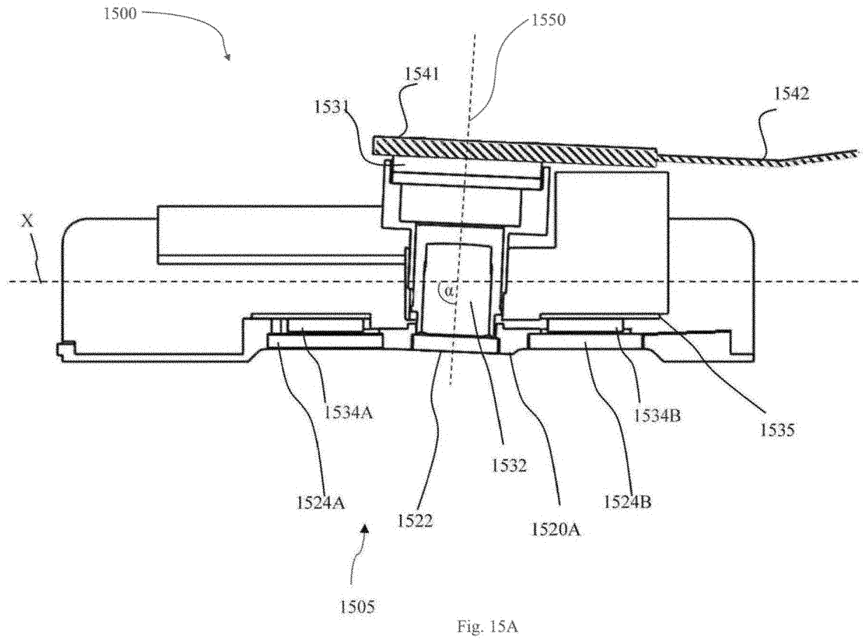

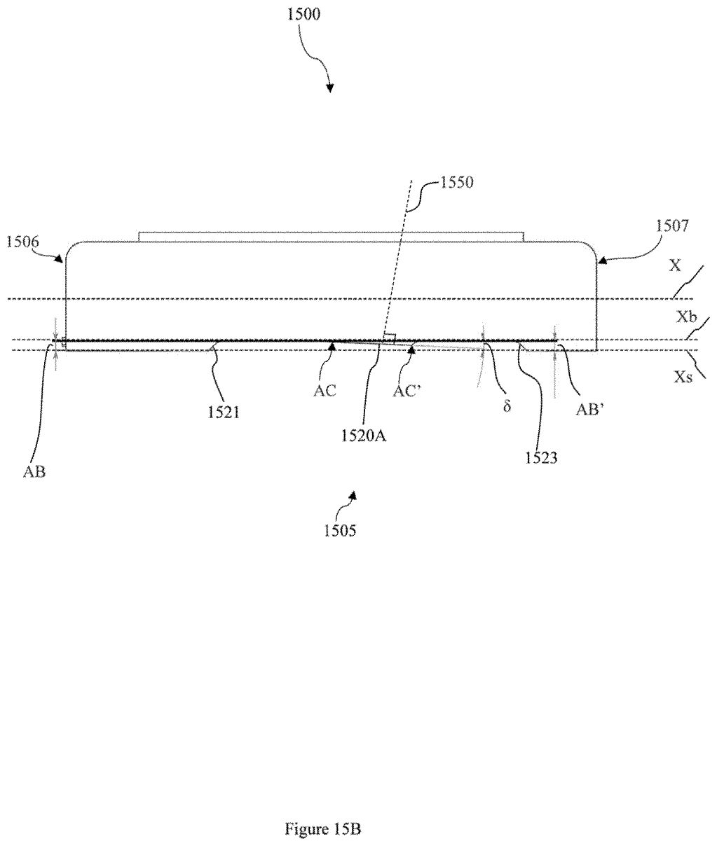

FIG. 15A shows a secondary modular imaging unit of a distal tip, according to exemplary embodiments of the disclosed subject matter. FIG. 15A shows a secondary modular imaging unit 1500 adapted to interlock and connect with a primary modular imaging unit (not shown) and a front modular imaging unit (not shown) to form a distal tip (not shown), for example primary modular imaging unit 1220, front modular imaging unit 1260 and secondary modular imaging unit 1240 of a distal tip 1210 as described with reference to FIG. 12. The secondary modular imaging unit 1500 comprises a second side lens assembly 1532 coupled with a second side sensor 1531 configured to receive the light captured by the second side lens assembly 1532 and convert that light to electric signals in a form of current. The secondary modular imaging unit 1500 further comprises a second side rigid circuit board 1541 configured to communicate with some optical gear components required for the operation of the second side lens assembly 1532, which in some cases may reside within a camera. In some cases, the second side rigid circuit board 1541 can be connected to a second side longitudinal circuit board section 1542 extending from the second side rigid circuit board 1541 to a rigid shaft (not shown).

The secondary modular imaging unit 1500 may comprise a second side niche 1505 positioned on a lateral side of the secondary modular imaging unit 1500 along the secondary modular imaging unit 1500 longitudinal axis X and designed to house a second side camera optical window 1522 and allows emission of light from second side illuminator optical windows 1524A and 1524B for illuminating the area captured by the second side lens assembly 1532 and second side sensor 1531. The second side niche 1505 may further comprises a second side tilted camera platform 1520A designed to tilt/elevate, in a predesign angle at least one component of the second side camera. The second side tilted camera platform 1520A can be designed to house the second side lens assembly 1532 in a tilted fashion, wherein a direction of view 1550 of the second side lens assembly 1532 forms a side angle α with the secondary modular imaging unit 1500 longitudinal axis X. The longitudinal axis X is defined as an axis extending parallelly to the distal tip (not shown). The direction of view 1550 of the second side lens assembly 1532 is defined as a straight and imaginary line extending from the center of the second side lens assembly 1532 through the focal point of the second side lens assembly 1532. In some cases, the side angle α may be in the range between 45 to 90 degrees, in the range of 75 to 90 degrees, in the range of 85 to 90 degrees. In such cases, the longitudinal axis X extends in parallel with the direction of view of a front lens assembly of a front modular imaging unit (not shown) of the distal tip (not shown). Thus, the side angle α is formed by the direction of view of the second side lens assembly 1532 and the direction of view of the front lens assembly (not shown).

In some embodiments of the disclosed subject matter, the second side tilted camera platform 1520A is designed to house the second side lens assembly 1532 in a tilted fashion and thereby allowing to form overlapping views between the second side lens assembly 1532 and the front lens assembly. The formed overlapping view's can allow capturing one object simultaneously from the front and second side cameras. Thus, the second side tilted camera platform 1520A may allow to generate one continues and panoramic view out of the views captured by more than one camera. In some cases, the front lens assembly may also form overlapping views with another side lens assembly, for example a first side lens assembly (not shown) of a primary modular imaging unit (not shown) of the distal tip (not shown). In such cases, the second side tilted camera platform 1520A can be the enabler forming one continues and panoramic view out of three lens assemblies.

In some cases, second side lens assembly 1532 resides within the front camera. Such, as the second side tilted camera platform 1520A positions in a dedicated tilted structure the second side camera optical window 1522 of the second side camera may be positioned in a tilt position, also, as the second side tilted camera platform 1520A positions in a dedicated tilted structure the second side camera optical window 1522 and the second side lens assembly 1532 of the second side camera may be positioned in a tilt position also, as the second side tilted camera platform 1520A positions in a dedicated tilted structure the second side camera optical window 1522, the second side lens assembly 1532 and the second side sensor 1531 of the second side camera may be positioned in a tilt position, moreover, as the second side tilted camera platform 1520A positions in a dedicated tilted structure the second side camera optical window 1522, the second side lens assembly 1532 and the second side sensor 1531 of the second side camera, along with the second side rigid circuit board 1541 may be positioned in a tilt position. Thus, the second side tilted camera platform 1520A can situate at least one of the second side camera components in a position wherein the imagery straight line following the direction of view of the second side lens assembly 1532 and the imagery straight line following direction of view of the front lens assembly (not shown) may be non-perpendicularly. For example, overlapping views may be formed by the view captured by the second side lens assembly 1532 and the view captured by the front lens assembly (not shown). The second side lens assembly 1532 and the front lens assembly may be adapted to provide the overlapping views at the working distance. In some cases, the tilt of the second side lens assembly 1532 may also enable the overlapping views between the front camera and the second side camera to align with the overlapping views between the front camera and the first side camera, wherein the first side camera placed in a first distance from the front camera and the second side camera placed in a second distance from the front camera and wherein the first distance is shorter than the second distance.

The secondary modular imaging unit 1500 further comprises second side illumination modules 1534A and 1534B residing in a second side illuminator electronic board 1535. The second side illuminator electronic board 1535 is situated within the secondary modular imaging unit 1500 such that the second side illumination modules 1534A and 1534B are abutted to the second side illuminator optical windows 1524A and 1524B respectively. In some cases, the number and location of the second side illumination modules 1534A and 1534B may vary for example, less than two first side illumination modules or more, wherein each of the second side illumination modules 1534A and 1534B may hold 1, 2, 3, 4 or more LED. The second side illumination modules 1534A and 1534B are configured to emit light, at the same light spectrum or at different light spectrum, through the second side illuminator optical windows 1524A and 1524B for allowing the second side camera to receive light from/in a second side field of view.

FIG. 15B shows a schematic cross-sectional side view of a secondary modular imaging unit of a distal tip, according to FIG. 15A. FIG. 15B shows a secondary modular imaging unit 1500 adapted to be connected to a distal tip (not shown). The secondary modular imaging unit 1500 has a U-shaped design having a proximal end 1506 and a distal end 1507, the secondary modular imaging unit 1500 comprises a second side niche 1505 positioned on a lateral side of the secondary modular imaging unit 1500 along the secondary modular imaging unit 1500 longitudinal axis X. The second side niche 1505 extends between a niche side all 1521 and a niche side wall 1523 characterized with niche depths, niche depth AB and niche depth AB′, respectively. In some embodiments of the disclosed subject matter, niche depth AB and niche depth AB′ may be the depth relatively to second side niche 1505 bottom, represented by axis Xb, and second side niche 1505 outer edge, represented by axis Xs. Wherein typically, second side niche 1505 outer edge may also be a secondary external surface of the secondary modular imaging unit 1500, for example secondary external surface 1243A of the secondary modular imaging unit 1240 of a distal tip 1210 as described with reference to FIG. 12. In some embodiments, the second side niche 1505 may comprise a uniform depth throughout the entire length of the second side niche 1505. In such cases, depths AB and AB′ may be identical. In some exemplary cases, the unitary depth of the second side niche 1505 may be in the range of 0.01-1.00 millimeter. In further exemplary cases, the unitary depth of the second side niche 1505 may be in the range of essentially 0.05-1.00 millimeter and in even further embodiments, in the range of 0.1-0.8 millimeter. In other cases, depth AB and depth AB′may comprise different values, such niche depth AB may be in the range of 0.01 and 1.00 millimeters. In some possible of the disclosed subject matter, the niche depth AB may be in the range of 0.05 and 1.00 millimeters and in even further embodiments, in the range of 0.1 to 0.8 millimeters. In some embodiments, the niche depth AB′ may be in the range of 0.01 to 1.00 millimeters. In some possible of the disclosed subject matter, the niche depth AB′ may be in the range of between 0.05 and 1.00 millimeters and in even further embodiments, in the range of 0.1-0.8 millimeters.

The second side niche 1505 may comprise a second side tilted camera platform 1520A having a proximal/front end AC and a distal/rear end AC′. In some embodiments of the disclosed subject matter, the proximal end AC has a depth in the range of 0.00 to 0.01 millimeters, in the range of 0.00 to 0.1 millimeters relatively to second side niche 1505 outer edge axis Xs. In some possible embodiments of the disclosed subject matter, the distal end AC′ may be tilted/elevated relatively to second side niche 1505 bottom axis Xb in an angle δ of about 0.5 to 10 degrees, of about 0.5 to 4.5 degrees, of about 1.5 to 3.5 degrees, of about 2.0 to 3.0 degrees.

As shown, a direction of view 1550 extends perpendicularly to second side tilted camera platform 1520A. As aforementioned, the direction of view 1550 is an imaginary line extending from a second side camera (not shown) of the second side optical gear (not shown) and through the center of the second side camera optical window (not shown) housed at the second side tilted camera platform 1520A. In some cases, the direction of view 1550 is further defined as the line extending through a second side field of view captured/received/observed by the second side camera (not shown), which separates the second side field of view into two equal parts. In some cases, the direction of view 1550 may be tilted/elevated in the angle δ from the second side niche 1505 bottom axis Xb toward the proximal end 1506 of the secondary modular imaging unit 1500. Thus, it is emphasized that the tilting of the direction of view 1550, and therefore the second side field of view provided by the second side camera is resulted by the tilting of the second side tilted camera platform 1520A.

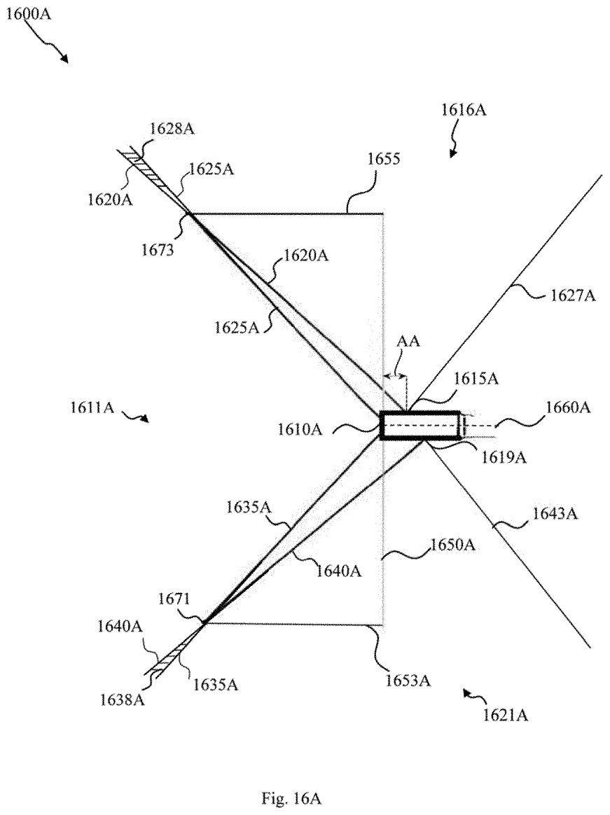

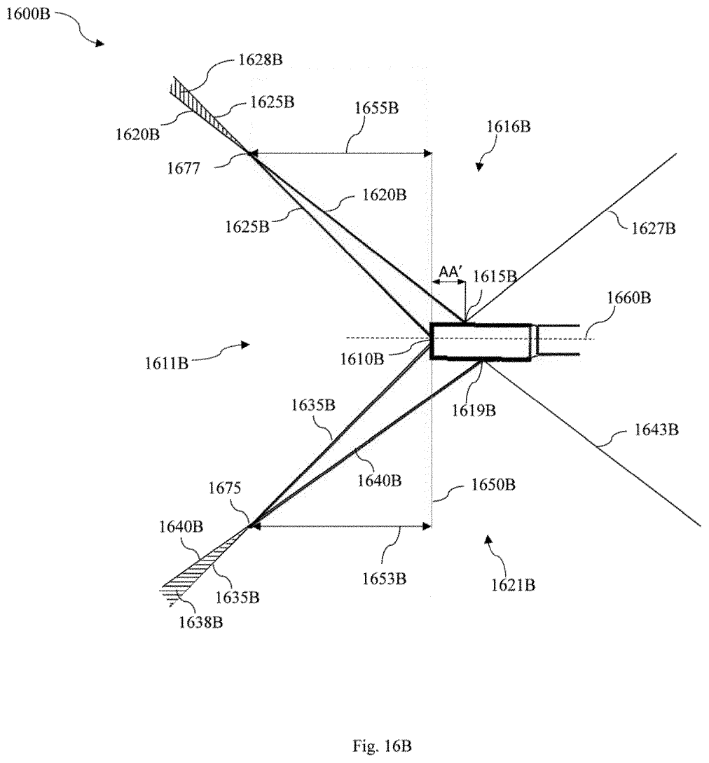

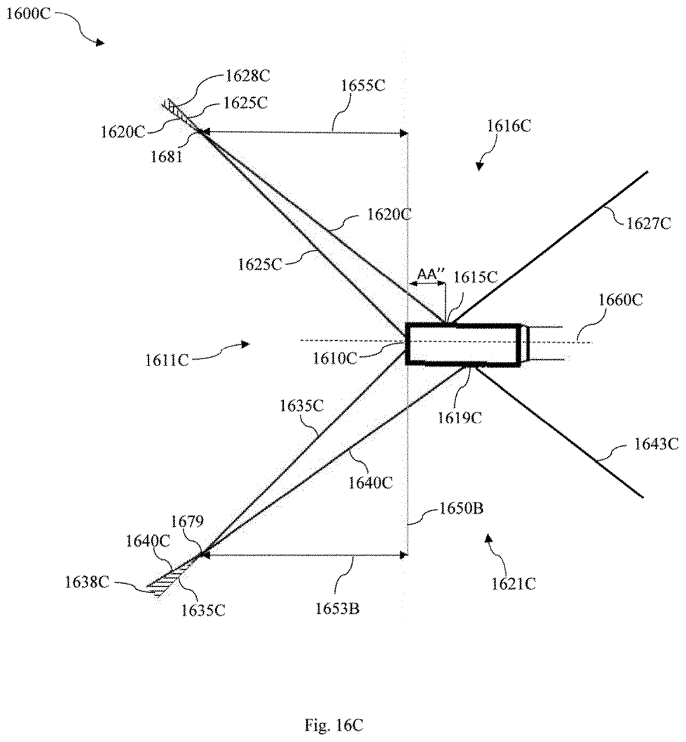

In some cases, the angle δ of the second side tilted camera platform 1520A may also enable overlapping views between a front camera (not shown) and the second side camera to align with the overlapping views between the front camera and a first side camera (not shown) wherein such alignment between the two overlapping views may provide a panoramic view. In some embodiments, the first side camera is placed in a first distance from the front camera and the second side camera is placed in a second distance from the front camera, wherein the first distance is shorter than the second distance. Another factor which influences the panoramic view is the field of view of each camera, such the front camera, first side camera and the second side camera. Such tilting/elevating second side camera to angle δ, might cause a first overlap point between the front camera FOV and the first side camera FOV to be equal or almost equal to a second overlap point between the front camera FOV and the second side camera. Also, the smaller the field of view of the front and each of the side cameras the longer the overlap point might be. Panoramic view at different FOVs is illustrated in FIG. 16A-16C.

FIG. 16A shows a schematic view of a multi camera medical imaging device with a front camera and two side cameras and wherein each of the front and two side cameras having lens assemblies creating a field of view of about 90 to 100 degrees, of about 95 degrees, according to exemplary embodiments of the disclosed subject matter. FIG. 16A shows a multi camera medical imaging device 1600A comprising a front camera with lens assembly 1610A characterized with a front field of view (FOV) 1611A representing the observable horizontal field of view of the front camera with lens assembly 1610A and spanned between an imaginary line 1625A and imaginary line 1635A. The front camera with lens assembly 1610A is also characterized with a direction of view 1660A defined as a straight and imaginary line extending from the center of the front camera with lens assembly 1610A through the focal point of the lens assembly 1610A of the front camera.

The multi camera medical imaging device 1600A also comprises a first side camera with lens assembly 1615A, wherein a distance AA between a center of first lens assembly 1615A and a front end of the multi camera medical imaging device 1600A may be approximately 10 to 17 millimeters. In some other cases, the distance AA between the center of first side lens assembly 1615A and the front end may be approximately 4 to 15 millimeters. The first camera with lens assembly 1615A also characterized with a first side field of view (FOV) 1616A representing the observable horizontal field of view captured by the front lens assembly 1615A and spanned between an imaginary line 1620A and imaginary line 1627A. The first side field of view 1616A can be characterized with an opening angle of essentially 90 to 100 degrees, essentially 95 degrees such that, the first side field of view 1616A and the front field of view 1611A are overlapping views, in case both fields of view share a field of view defined as overlapped view 1628A. Thus, an object seen at the overlapped view 1628A may be captured simultaneously by the first side camera comprising the first lens assembly 1615A and the front camera comprising the front lens assembly 1610A. In such cases, the seen object can be captured in dissimilar angles by each lens assembly. The overlapped view 1628A may be characterized with a first overlap point 1673 defines a first overlap distance 1655A in which the front field of view 1611A crosses the first side field of view 1616A. In some cases, the first overlap point 1673 defines the first overlap distance 1655A in which the imaginary line 1625A representing the boundary of the front field of view 1611A, crosses the imaginary line 1620A representing the boundary of the first side field of view 1616A. The first side camera comprising the first lens assembly 1615A further characterized with a direction of view defined as a straight and imaginary line extending from the center of the first lens assembly 1615A through the focal point of the first lens assembly 1615A, wherein the first side camera direction of view is typically perpendicularly to the front camera direction of view 1660A.

In some embodiments of the disclosed subject matter an opening angle of essentially 90 to 100 degrees, essentially 95 degrees of the first side field of view 1616A and an opening angle of about 90 to 100 degrees, of about 95 degrees of the front field of view 1611A may determine the first overlap point 1673 defines the first overlap distance 1655A in a range of 78 to 90 millimeters from an imaginary line 1650A, wherein the imaginary line 1650A is tangent to a front lens optical window of the front lens assembly 1610A of the front camera, and typically perpendicularly to the direction of view 1660A.

The multi camera medical imaging device 1600A also comprises a second camera comprising a second side lens assembly 1619A, wherein the second side lens assembly 1619A is placed within a second side tilted camera platform (not shown), for example second side tilted camera platform 1330A of a second side niche 1330 of a distal tip 1305 as described with reference to FIGS. 13A-13B. In some cases, the distance between a center of second side camera 1619A and the center of first side camera 1615A is between 0 to 10 millimeters. The second side camera comprising the second side lens assembly 1619A also characterized with a second side field of view (FOV) 1621A representing the observable horizontal view of the second side lens assembly 1619A and spanned between an imaginary line 1640A and an imaginary line 1643A. The second side field of view 1621A can be characterized with an opening angle of 90 to 100 degrees, essentially 95 degrees such that, second side field of view 1621A and the front field of view 1611A are overlapping views, in case both fields of view share a field of view defined as overlapped view 1638A. Thus, an object seen at the overlapped view 1638A may be captured simultaneously by the front camera comprising the front lens assembly 1610A and the second side camera comprising the second side lens assembly 1619A. In such cases, the seen object can be captured in dissimilar angles by each lens assembly. The overlapped view 1638A may be characterized with a second overlap point 1671 defines the second overlap distance in which the front field of view 1611A crosses the second side field of view 1621A. In some cases, the second overlap point 1671 defines the second overlap distance in which the imaginary line 1635A representing the boundary of the front field of view 1611A, crosses the imaginary line 1640A representing the boundary of the second side field of view 1621A. The second side lens assembly 1619A of the second side camera further characterized with a direction of view defined as a straight and imaginary line extending from the center of the second side camera 1619A through the focal point of the second side lens assembly 1619A, wherein the second side lens assembly 1619A direction of view is typically non-perpendicularly to the front direction of view 1660A due to the tilted position of the second side camera comprising the direction of view 1660A, 1619A within the second side tilted camera platform.

In some embodiments of the disclosed subject matter the opening angle of essentially 90 to 100 degrees, essentially 95 degrees of the second side field of view 1621A and the opening angle of about 90 to 100 degrees, of about 95 degrees of the front field of view 1611A may determine the second overlap point 1675 defines a second overlap distance 1653A which is in a range of between 78 to 85 millimeters from the imaginary line 1650A.

Thus, in some embodiments, wherein the front field of view 1611A, the first side field of view 1616A and the second side field of view 1621A are about 90 to 100 degrees, of about 95 degrees, and wherein the distance between a center of second side lens assembly 1619A and the center of first side lens assembly 1615A is not 0 millimeters and wherein the second side camera is placed within the second side tilted camera platform, a surround view may be provided at first overlap distance 1655A of about 80 to 90 millimeters and second overlap distance 1653A ckf about 78 to 85 millimeters.

FIG. 16B shows a schematic view of multi camera medical imaging device with a front camera comprising a front lens assembly with a field of view characterized with an opening angle of between 88 to 98 degrees and two side cameras comprising side lens assemblies each with field of views characterized with an opening angle of about 90 to 110 degrees, of about 104 degrees, according to exemplary embodiments of the disclosed subject matter. FIG. 16B shows a multi camera medical imaging device 1600B comprising a front camera comprising a front lens assembly 1610B characterized with a front field of view 1611B representing the observable horizontal field of view captured by the front lens assembly 1610B and spanned between an imaginary line 1625B and imaginary line 1635B. The front lens assembly 1610B can be characterized with an opening angle of essentially 88 to 98 degrees. In some cases, the front lens assembly 1610B can be characterized with an opening angle of essentially 90 degrees. In some cases, the front lens assembly 1610B can be characterized with an opening angle of essentially 95 degrees. The front lens assembly 1610B is also characterized with a direction of view 1660B defined as a straight and imaginary line extending from a center of the front lens assembly 1610B through the focal point of the front lens assembly 1610B, typically, direction of view 1660B is parallel to multi camera medical imaging device 1600B longitudinal axis (not shown).

The multi camera medical imaging device 1600B also comprises a first side camera comprising a first side lens assembly 1615B wherein a distance AA′ between a center of first side lens assembly 1615B and a front end of the multi camera medical imaging device 1600B may be approximately 10 to 17 millimeters. In some other cases, the distance AA′ between the center of first side camera comprising the first side lens assembly 1615B and the front end may be approximately 4 to 15 millimeters. The first side camera comprising the first lens assembly 1615B also characterized with a first side field of view 1616B representing the observable horizontal field of view captured by the front lens assembly 1615B and spanned between an imaginary line 1627B and an imaginary line 1620B. The first side field of view 1616B can be characterized with an opening angle of essentially 100 to 110 degrees, essentially 104 degrees such that, the first side field of view 1616B are overlapping views, in case both fields of view share a field of view defined as overlapped view 1628B. Thus, an object seen at the overlapped view 1628B may be captured simultaneously by the first side camera comprising the first side lens assembly 1615B and the front side camera comprising the front lens assembly 1610B. In such cases, the seen object can be captured in dissimilar angles by each lens assembly. The overlapped view 1628B may be characterized with a first overlap point 1677 defines a first overlap distance 1655B in which the front field of view 1611B crosses the first side field of view 1616B. In some cases, the first overlap point 1677 defines the first overlap distance 1655B in which the imaginary line 1625B representing the boundary of the front field of view 1611B, crosses the imaginary line 1620B representing the boundary of the first side field of view 1616B. The first side camera comprising the first side lens assembly 1615B further characterized with a direction of view defined as a straight and imaginary line extending from the center of the first lens assembly 1615B through the focal point of the first side lens assembly 1615B, wherein the first side lens assembly direction of view is typically perpendicularly to the direction of view 1660B of the front lens assembly 1610B.

In some embodiments of the disclosed subject matter the opening angle of essentially 104 degrees of the first side field of view 1616B and the opening angle of about 90 degrees of the front field of view 1611B may determine the first overlap point 1677 to be distant in a range of 60 to 64 millimeters from an imaginary line 1650B, wherein the imaginary line 1650B is tangent to a front lens optical window of the front camera comprising the front lens assembly 1610B, and typically perpendicularly to the direction of view 1660B. In some embodiments of the disclosed subject matter, the opening angle of essentially 104 degrees of the first side field of view 1616B and the opening angle of about 95 degrees of the front field of view 1611B may determine the first overlap point 1677 to be distant in a range of 42 to 48 millimeters from the imaginary line 1650B. The multi camera medical imaging device 1600B also comprises a second side camera comprising a second side lens assembly 1619B, wherein the second side lens assembly 1619B is placed within a second side tilted camera platform (not shown), for example second side tilted camera platform 1330A of a second side niche 1330 of a distal tip 1305 as described with reference to FIGS. 13 A-13B. In some cases, the distance between a center of second side lens assembly 1619B and the center of first side lens assembly 1615B is between 0 to 10 millimeters. The second side lens assembly 1619B also characterized with a second side field of view 1621B representing the observable horizontal view of the second side lens assembly 1619B and spanned between an imaginary line 1640B and an imaginary line 1643B. The second side field of view 1621B can be characterized with an opening angle of essentially 100 to 110 degrees, essentially 104 degrees such that, second side field of view 1621B and the front field of view 1611B are overlapping views, in case both fields of view share a field of view defined as overlapped view 1638B. Thus, an object seen at the overlapped view 1638B may be captured simultaneously by the second side field of view 1619B and the front lens assembly 1610B. In such cases, the seen object can be captured in dissimilar angles by each lens assembly. The overlapped view 1638B may be characterized with a second overlap point 1675 defines a second overlap distance 1653B in which the front field of view 1611B crosses the second side field of view 1621B. In some cases, the second overlap point 1675 defines the second overlap distance in which the imaginary line 1635B representing the boundary of the front field of view 1611B, crosses the imaginary line 1640B representing the boundary of the second side field of view 1621B. The second side lens assembly 1619B further characterized with a direction of view defined as a straight and imaginary line extending from the center of the second side lens assembly 1619B through the focal point of the second side lens assembly 1619B, wherein the second side lens assembly 1619B direction of view is typically non-perpendicularly to the front camera direction of view 1660B due to the tilted position of the second side camera comprising the second side lens assembly 1619B within the second side tilted camera platform.

In some embodiments of the disclosed subject matter the opening angle of essentially 104 degrees of the second side field of view 1619B and the opening angle of about 90 degrees of the front field of view 1611B may determine the second overlap point 1675 defines the second overlap distance 1653B from an imaginary line 1650B to be in a range of about 60 to 64 millimeters from the imaginary line 1650B. In some embodiments of the disclosed subject matter, the opening angle of essentially 104 degrees of the second side field of view 1619B and the opening angle of 95 degrees of the front field of view 1611B may determine the second overlap point 1675 to be distant in a range of 42 and 48 millimeters from the imaginary line 1650B.

Thus, in some embodiments, wherein the front field of view 1611B is about 90 degrees, and the first side field of view 1616B and the second side field of view 1621B are about 104 degrees, and wherein the distance between a center of second side lens assembly 1619B and the center of first side lens assembly 1615B is not 0 millimeters and wherein the second side camera comprising the second side lens assembly 1619B is placed within the second side tilted camera platform, a surround view may be provided at first overlap distance 1655B of about 60 to 64 millimeters and second overlap distance 1653B of about 60 to 64 millimeters. In other embodiments, wherein the front field of view 1611B is about 95 degrees, and the first side field of view 1616B and the second side field of view 1621B are about 104 degrees, and wherein the distance between a center of second side lens assembly 1619B and the center of first side lens assembly 1615B is not 0 millimeters and wherein the second side camera comprising the second side lens assembly 1619B is placed within the second side tilted camera platform, a surround view may be provided at first overlap distance 1655B of about 42 to 48 millimeters and second overlap distance 1653B of about 42 to 48 millimeters.

FIG. 16C shows a schematic view of multi camera medical imaging device representing a number of conditional implementations of a front camera comprising a front lens assembly and two side cameras each side camera comprises a side lens assembly, according to exemplary embodiments of the disclosed subject matter. FIG. 16C shows a multi camera medical imaging device 1600C comprising a front camera 1610C characterized with a front field of view 1611C. representing a section of the observable horizontal field of view captured by the front lens assembly 1610C and spanned between an imaginary line 1625C and an imaginary line 1635C. The front lens assembly 1610C is also characterized with a direction of view 1660C defined as a straight and imaginary line extending from the center of the front lens assembly 1610C through the focal point of the front lens assembly 1610B, typically, direction of view 1660C is parallel to multi camera medical imaging device 1600C longitudinal axis (not shown).

The multi camera medical imaging device 1600C also comprises a first lens assembly 1615C, wherein a distance AA″ between a center of first side lens assembly 1615C and a front end of the multi camera medical imaging device 1600C may be approximately 10 to 17 millimeters. In some other cases, the distance AA″ between the center of first side lens assembly 1615C and the front end may be approximately 4 to 15 millimeters. The first side lens assembly 1615C also characterized with a first side field of view 1616C representing the observable horizontal field of view of a camera comprising first side lens assembly 1615C and spanned between an imaginary line. 1627C and an imaginary line 1620C. The first side field of view 1616C can be characterized with an opening angle such that the first side of view 1616C and the front field of view 1611C are overlapping views, in case both fields of view share a field of view defined as overlapped view 1628C. Thus, an object seen at the overlapped view 1628C may be captured simultaneously by the first lens assembly 1615C and the front lens assembly 1610C. In such cases, the seen object can be captured in dissimilar angles by each lens assembly. The overlapped view 1628C may be characterized with a first overlap point 1681 defines a first overlap distance 1655C in which the front field of view 1611C crosses the first side field of view 1616C. In some cases, the first overlap point 1681 defines the first overlap distance 1655C in which the imaginary line 1625C representing the boundary of the front field of view 1611C, crosses the imaginary line 1620C representing the boundary of the first side field of view 1616C. The first side lens assembly 1615C further characterized with a direction of view defined as a straight and imaginary line extending from the center of the first side lens assembly 1615C through the focal point of the first side lens assembly 1615C, wherein the first side lens assembly direction of view is typically perpendicularly to the front lens assembly direction of view 1660C.

The multi camera medical imaging device 1600C also comprises a second side lens assembly 1619C, wherein a second side camera comprising the second side lens assembly 1619C is placed within a second side tilted camera platform (not shown), for example second side tilted camera platform 1330A of a second side niche 1330 of a distal tip 1305 as described with reference to FIGS. 13A-13B. In some cases, the distance between a center of second side lens assembly 1619C and the center of first side lens assembly 1615C is between 0 to 10 millimeters. The second side lens assembly 1619C also characterized with a second side field of view 1621C representing the observable horizontal view of the second side lens assembly 1619C and spanned between an imaginary line 1640C and an imaginary line 1643C. The second side field of view 1621C can be such that the second side field of view 1621C and the front field of view 1611C are overlapping views, in case both fields of view share a field of view defined as overlapped view 1638C. Thus, an object seen at the overlapped view 1638C may be captured simultaneously by both lens assemblies, the second side field of view 1619C and the front lens assembly 1610C. In such cases, the seen object can be captured in dissimilar angles by each lens assembly. The overlapped view 1638C may be characterized with a second overlap point 1679 defines a second overlap distance 1653C in which the front field of view 1611C crosses the second side field of view 1621C. In some cases, the second overlap point 1679 may be characterized as the point in which the imaginary line 1635C representing the boundary of the front field, of view 1611C, crosses the imaginary line 1640C representing the boundary of the second side field of view 1621C.

The second side camera comprises a second side lens assembly 1619C further characterized with a direction of view defined as a straight and imaginary line extending from the center of the second side lens assembly 1619C through the focal point of the second side lens assembly 1619C, wherein the second side lens assembly 1619C direction of view is typically non-perpendicularly to the front lens assembly direction of view 1660C due to the tilted position of the second side lens assembly 1619C within the second side tilted camera platform.

In some embodiments of the disclosed subject matter, an opening angle of about 100 to 106 degrees of the first side field of view 1616C, and an opening angle of about 100 to 106 degrees of the front field of view 1611C may determine the first overlap point 1681 defines the first overlap distance 1655C from an imaginary line 1650C to be in a range of about 23 to 33 millimeters from the imaginary line 1650C. In some embodiments of the disclosed subject matter, an opening angle of about 100 to 106 degrees of the second side field of view 1619C, and an opening angle of about 100 to 106 degrees of the front field of view 1611C may determine the second overlap point 1679 defines the second overlap distance 1653C from an imaginary line 1650C to be in a range of about 23 to 33 millimeters from the imaginary line 1650C.

Thus, in some embodiments, wherein the front field of view 1611C is about 104 degrees, and the first side field of view 1616C and the second side field of view 1621C are about 104 degrees, and wherein the distance between a center of second side lens assembly 1619C and the center of first side lens assembly 1615C is not 0 millimeters and wherein the second side lens assembly 1619C is placed within the second side tilted camera platform, a surround view may be provided at the first overlap distance 1655C of about 23 to 33 millimeters and second overlap distance 653C of about 23 to 33 millimeters.

In some embodiments of the disclosed subject matter, an opening angle of about 90 to 106 degrees of the first side field of view 1616C, and an opening angle of about 90 to 106 degrees of the front field of view 1611C may determine the first overlap point 1681 defines the first overlap distance 1655C from an imaginary line 1650C to be in a range of about 23 to 90 millimeters from the imaginary line 1650C. In some embodiments of the disclosed subject matter, an opening angle of about 90 to 106 degrees of the second side field of view 1619C, and an opening angle of about 90 to 106 degrees of the front field of view 1611C may determine the second overlap point 1679 defines the second overlap distance 1653C from an imaginary line 1650C to be in a range of about 23 to 90 millimeters from the imaginary line 1650C.

While the disclosure has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings without departing from the essential scope thereof. Therefore, it is intended that the disclosed subject matter not be limited to the particular embodiment disclosed as the best mode contemplated for carrying out this invention, but only by the claims that follow.

1. A multi camera medical imaging device, comprising:
a distal tip adapted to be connected to a rigid shaft of the multi camera imaging device, comprising:
a front modular imaging unit comprising a front optical gear, wherein the front optical gear captures a front field of view;
a primary modular imaging unit adapted to house the front modular imaging unit, wherein said primary modular imaging unit comprises a longitudinal opening located on a lateral side of the primary modular imaging unit, in parallel to a longitudinal axis of the primary modular imaging unit;
a secondary modular imaging unit comprising a second side optical gear, wherein the second side optical gear captures a second side field of view, wherein the second side optical gear is located in parallel to a longitudinal axis of the secondary modular imaging unit, and wherein said secondary modular imaging unit is adapted to close the longitudinal opening of said primary modular imaging unit and function as a bracket which seals the longitudinal opening.

2. The multi camera medical imaging device of claim 1, wherein the primary modular imaging unit further comprises a first side optical gear configured to capture a first side field of view required for the operation of the multi camera medical imaging device.

3. The multi camera medical imaging device of claim 2, wherein a first side foldable circuit board of the primary modular imaging unit comprising a first side longitudinal circuit board positioned parallel to the longitudinal axis and extends outward from a rear ed section of primary modular imaging unit into the rigid shaft and a foldable arm configured to a foldable position, and to connect a first side rigid circuit board to the first side longitudinal circuit board, wherein the first side rigid circuit board designed to carry a first side camera, and wherein the first side foldable circuit board configured to locate the first side optical gear within the primary modular imaging unit.

4. The multi camera medical imaging device of claim 1, wherein the secondary modular imaging unit comprises protruding elements designed to connect and fasten the secondary modular imaging unit into the primary modular imaging unit.

5. The multi camera medical imaging device of claim 4, wherein the protruding elements are located at margins of edge of the secondary modular imaging unit, wherein said edge has a U-shape adapted to interlock within the longitudinal opening of the primary modular imaging unit.

6. The multi camera medical imaging device of claim 1, wherein a second side foldable circuit board of the secondary modular imaging unit comprising a second side longitudinal circuit board positioned in parallel to the modular imaging unit longitudinal axis and extends outward from a rear end section of secondary modular imaging unit into the primary modular imaging unit and into the rigid shaft, and a second side rigid circuit board connected to the second side longitudinal circuit board and designed to carry a second side camera wherein the second side foldable circuit board configured to locate the second side optical gear within the secondary modular imaging unit.

7. (canceled)

8. (canceled)

9. (canceled)

10. (canceled)

11. The multi camera medical imaging device of claim 1, wherein the primary modular imaging unit further comprises a first side illuminator electronic circuit board designed to host first side illumination modules.

12. (canceled)

13. (canceled)

14. The multi camera medical imaging device of claim 1, wherein the secondary modular imaging unit further comprises one or more protruding element located at margins of the secondary modular imaging unit to be inserted into corresponding openings in the primary modular imaging unit.

15. The multi camera medical imaging device of claim 1, wherein a front foldable circuit board of the front modular imaging unit comprising
a front longitudinal circuit board positioned in parallel to the primary modular imaging unit longitudinal axis and extends outward from the primary modular imaging unit into the rigid shaft; and
a front foldable arm configured to a foldable position and to connect a front rigid circuit board to the front longitudinal circuit board, wherein the front rigid circuit board designed to carry a front camera.

16. (canceled)

17. (canceled)

18. A multi camera medical imaging device, comprising:
a distal tip adapted to be connected to a rigid shaft of the multi camera imaging device, comprising:
a front modular imaging unit comprising a front optical gear, wherein the front optical gear comprises a front camera and a front illumination module configured to capture a front field of view;
a primary modular imaging unit comprising a first side optical gear, wherein the first side optical gear comprises a first side camera positioned such to have a first direction of view, wherein the first side camera is further tilted with a first side angle configured to capture a first side field of view, wherein the first direction of view is adapted to provide an overlapped view between said front field of view and the first direction of view at a first overlap distance;
a secondary modular imaging unit comprising a second side optical gear, wherein the second side optical gear comprises a second side camera position such to have a second direction of view, wherein the second side camera is further tilted with a second side angle configured to capture a second side field of view, wherein the second direction of view is adapted to provide an overlapped view between said front field of view and the second direction of view at a second overlap distance wherein the tilt of the second camera is configured to provide the overlapping view between the front camera and the second side camera to align with the overlapping view between the front camera and the first side camera wherein said first overlap distance equals to said second overlap distance.

19. The multi camera medical imaging device of claim 18, wherein the first side angle,
which is the angle between the first side camera and a longitudinal axis of the distal tip, is between 85 degrees to 90 degrees.

20. (canceled)

21. The multi camera medical imaging device of claim 18, wherein said secondary modular imaging unit further comprises a second side niche located on an external surface of the secondary modular imaging unit parallel to the secondary modular imaging unit longitudinal axis and configured to house a second side camera optical window abutted to the second side camera and second side illuminator optical windows abutted to second side illumination modules.

22. The multi camera medical imaging device of claim 21, wherein the second side niche further comprises a second side tilted camera platform elevated in the second side angle from the secondary modular imaging unit longitudinal axis toward a distal front end of the front modular imaging unit and configured to house the second side camera optical window and the second side camera to provide the second side direction of view, wherein the second side direction of view extends perpendicularly to the second side tilted camera platform.

23. The multi camera medical imaging device of claim 21, wherein the secondary modular imaging unit further comprises
a second side foldable circuit board comprising
a second side longitudinal circuit board positioned in parallel to the distal tip longitudinal axis and extends outward from a rear end section of secondary modular imaging unit into the primary modular imaging unit and into the rigid shaft, and
a second side rigid circuit board connected to the second side longitudinal circuit board and configured to position a second side lens assembly and a second side sensor of the second side camera in the second side angle relatively to the distal tip longitudinal axis
wherein the second side foldable circuit board configured to locate the second side optical gear within the secondary modular imaging unit
wherein the secondary modular imaging unit further comprises a protruding element designed to connect and fasten the secondary modular imaging unit into the primary modular imaging unit.

24. (canceled)

25. (canceled)

26. A distal tip of a multi camera medical imaging device comprising
a first side lens assembly placed in a first distance from a distal front end of the distal tip and tilted to a first angle adapted to provide an overlap view between said first side lens assembly and a front lens assembly at a first overlap distance, and a second side lens assembly placed in a second distance from the distal front end of the and tilted to a second angle adapted to provide an overlap view between said second side lens assembly and the front lens assembly at a second overlap distance, wherein the first distance is shorter than the second distance, wherein the first overlap distance is equal to second overlap distance.

27. The multi camera medical imaging device of claim 15, wherein front foldable arm is configured to be fold with an angle between 0 to 95 degrees to the primary modular imaging unit longitudinal axis.

28. The multi camera medical imaging device of claim 15, wherein the first side camera placed in a first distance from the front camera and the second side camera placed in a second distance from the front camera and wherein the first distance is shorter than the second distance.

29. The multi camera medical imaging device of claim 18, wherein the second side angle, which is the angle between the second side camera direction of view and the longitudinal axis of the distal tip, is between 85 degrees to 90 degrees.

30. The multi camera medical imaging device of claim 18, wherein the first side camera placed in a first distance from a distal front end of the front modular imaging unit and wherein the second side camera placed in a second distance from the distal front end of the front modular imaging unit, wherein the first distance is shorter than the second distance.

31. The multi camera medical imaging device of claim 18, wherein the first side angle, which is the angle between the first side camera direction of view and the longitudinal axis of the distal tip, is between 85 degrees to 90 degrees.
